SEQUENCE LISTING (15 sequences)

SEQ ID NO: 1 - protein, 284 aa, Homo sapiens
QAAAPPKAVLKLEPPWINVLQEDSVTLTCQGARSPESDSIQWFHNGNLIPTHTQPSYRFKANNNDSGEYTCQTGQTSLSDPVHLTVLSEWLVLQTPHLEFQEGETIMLRCHSWKDKPLVKVTFFQNGKSQKFSHLDPTFSIPQANHSHSGDYHCTGNIGYTLFSSKPVTITVQVPSMGSSSPMGIIVAVVIATAVAAIVAAVVALIYCRKKRISANSTDPVKAAQFEPPGRQMIAIRKRQLEETNNDYETADGGYMTLNPRAPTDDDKNIYLTLPPNDHVNSNN

SEQ ID NO: 2 - protein, 248 aa, Homo sapiens
TPAPPKAVLKLEPQWINVLQEDSVTLTCRGTHSPESDSIQWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANPTNPDEADKVGAENTITYSLLMHPDALEEPDDQNRI

SEQ ID NO: 3 - protein, 281 aa, Homo sapiens
TPAAPPKAVLKLEPQWINVLQEDSVTLTCRGTHSPESDSIPWFHNGNLIPTHTQPSYRFKANNNDSGEYTCQTGQTSLSDPVHLTVLSEWLVLQTPHLEFQEGETIVLRCHSWKDKPLVKVTFFQNGKSKKFSRSDPNFSIPQANHSHSGDYHCTGNIGYTLYSSKPVTITVQAPSSSPMGIIVAVVTGIAVAAIVAAVVALIYCRKKRISANSTDPVKAAQFEPPGRQMIAIRKRQPEETNNDYETADGGYMTLNPRAPTDDDKNIYLTLPPNDHVNSNN

SEQ ID NO: 4 - protein, 11 aa, Homo sapiens
GASENIYGALN

SEQ ID NO: 5 - protein, 7 aa, Homo sapiens
GATNLAD

SEQ ID NO: 6 - protein, 9 aa, Homo sapiens
QNVLNTPLT

SEQ ID NO: 7 - protein, 10 aa, Homo sapiens
GYIFSNYWIQ

SEQ ID NO: 8 - protein, 17 aa, Homo sapiens
EILPGSGSTEYTENFKD

SEQ ID NO: 9 - protein, 13 aa, Homo sapiens
YFFGSSPNWYFDV

SEQ ID NO: 10 - protein, 107 aa, Homo sapiens [sequence omitted]

SEQ ID NO: 11 - protein, 122 aa, Homo sapiens [sequence omitted]

SEQ ID NO: 12 - protein, 214 aa, Homo sapiens [sequence omitted]

SEQ ID NO: 13 - protein, 448 aa, Homo sapiens
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 107 aa, Homo sapiens [sequence omitted]

SEQ ID NO: 15 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0076309 A1
(43) Pub. Date: Mar. 6, 2025

(54) FC-GAMMA RECEPTOR II BINDING AND GLYCAN CONTENT

(71) Applicant: AMGEN INC., Thousand Oaks, CA (US)

(72) Inventors: Palanisamy KANAKARAJ, North Andover, MA (US); Katariina HUTTERER, Newbury Park, CA (US); Scott KUHNS, Newbury Park, CA (US)

(73) Assignee: AMGEN INC., Thousand Oaks, CA (US)

(21) Appl. No.: 18/698,098

(22) PCT Filed: Oct. 4, 2022
(86) PCT No.: PCT/US2022/045633

§ 371 Date: Apr. 3, 2024

Related U.S. Application Data
(60) Provisional application No. 63/299,104, filed on Jan. 13, 2022.
(60) Provisional application No. 63/252,245, filed on Oct. 5, 2021.

(51) Int. Cl. G01N 33/68; C07K 16/18

(52) U.S. Cl. CPC G01N 33/6854; C07K 16/18; C07K 2317/41; C07K 2317/522; C07K 2317/524; C07K 2317/526; G01N 2400/10; G01N 2440/38

(57) ABSTRACT

Provided herein are methods of determining product quality of an antibody composition, wherein the product quality is based on the Fcγ receptor II (FcγRI) binding level of the antibody composition. In exemplary embodiments, the method comprises (a) determining the afucosylated glycan content and/or β-galactosylated glycan content of a sample of the antibody composition; (b) optionally, calculating a predicted FcγRII binding level based on the afucosylated glycan content and/or β-galactosylated glycan content as determined in (a); and (c) determining the product quality of the antibody composition as acceptable when (i) the afucosylated glycan content and/or β-galactosylated glycan content is within a target range and/or (ii) the predicted FcγRII binding level is within a target range. Related methods of monitoring product quality and methods of producing an antibody composition are further provided herein.

### CROSS REFERENCE TO RELATED APPLICATIONS

The benefit under 35 U.S.C. § 119(e) of U.S. Provisional Patent Application No. 63/252,245, filed on Oct. 5, 2021, and U.S. Provisional Patent Application No. 63/299,104 filed Jan. 13, 2022, is hereby claimed, and the disclosures thereof are hereby incorporated by reference herein.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 15.4 kilobyte XML file named “A-2755-WO01-SEC_Sequence_Listing.XML”; created on Sep. 22, 2022.

### BACKGROUND

Glycosylation is one of the most common, yet impactful, post-translational modifications (PTMs), as it plays a role in multiple cellular functions, including, for example, protein folding, quality control, molecular trafficking and sorting, and cell surface receptor interaction. Glycosylation affects the therapeutic efficacy of recombinant protein drugs, as it influences the bioactivity, pharmacokinetics, immunogenicity, solubility, and in vivo clearance of therapeutic glycoproteins. Fc glycoform profiles, in particular, are product quality attributes for recombinant antibodies, as they directly impact the clinical efficacy and pharmacokinetics of the antibodies.

Specific glycan structures associated with the conserved bi-antennary glycan in the Fc-CH2 domain can strongly influence the interaction of the Fc domain with the Fcrgamma receptors (FcγRs) that mediate antibody effector functions, e.g., antibody dependent cellular cytotoxicity (ADCC) (see Reusch D, Tejada M L. Fc glycans of therapeutic antibodies as critical quality attributes. Glycobiology 2015; 25:1325-34). For example, core fucose has been demonstrated to have a significant impact on FcγRIIIa binding affinity, leading to substantial changes in ADCC activity (see Okazaki A, et al. Fucose depletion from human IgG1 oligosaccharde enhances binding enthalpy and association rate between IgG1 and FcgammaRIIIa. Journal of molecular biology 2004; 336:1239-49; Ferrara C, et al. Unique carbohydrate-carbohydrate interactions are required for high affinity binding between FcgammaRIII and antibodies lacking core fucose. Proceedings of the National Academy of Sciences of the United States of America 2011; 108:12669-74). It has also been shown that high mannose levels play a role in modulating ADCC activity, though to a much more modest and less predictable extent than core fucose (Thomann M, et al. Fc-galactosylation modulates antibody-dependent cellular cytotoxicity of therapeutic antibodies. Molecular immunology 2016; 73:69-75). Because core fucose has been reported to sterically hinder the Fc domain from interacting with the FcγR, much research has focused on glycan groups which lack core fucose, including afucosylated glycans and high mannose glycans. In addition to these glycan groups which lack core fucose, terminal galactose has been suggested to influence ADCC levels. In particular the presence of terminal galactose enhances ADCC activity. Thomann et al., Molec Immunol 73: 69-75 (2016).

The structures of the glycans present on the antibody Fc domain can also impact Fc binding to complement protein C1q, and thus ultimately impacts the antibody's complement dependent cytotoxicity (CDC) effector function. For instance, antibodies with higher β-galactosylation bind to C1q with high affinity and induce higher levels of CDC activity. Similarly, reduced β-galactosylation of the anti-TNF antibody adalimumab associated with reduced ADCC activity and CDC activity. A decrease in β-galactosylation of adalimumab also associated with reduced binding affinity to FcγRIIIa binding and C1q protein. Burzawa et al., “Relationship between structure and function: Influence of galactosylation on Fc-mediated binding and functional properties of adalimumab” Bioprocess Online (2018) available at: https://www.bioprocessonline.com/doc/influence-of-galactosylation-on-fc-mediated-binding-and-functional-properties-of-adalimumab-0001.

Different factors influence the glycan structure and thus the ultimate glycosylated form (glycoform) of the protein (glycoprotein). For example, the cell line expressing the antibody, the cell culture medium, the feed medium composition, and the timing of the feeds during cell culture can impact the production of glycoforms of the protein. While research groups have suggested many ways to influence the levels of particular glycoforms of an antibody, there still is a need in the biopharmaceutical industry for simple and efficient methods to predict the level of effector function or binding to an FcγR a particular antibody composition will exhibit based on the given glycoform profile for that antibody composition. Additionally, there is a need in the art for methods of determining the levels of particular glycans that will achieve a desired level effector function or level of FcγR binding.

### SUMMARY

Provided herein for the first time are data demonstrating a statistically significant association between the FcγRII binding level of an antibody composition and the level of β-galactosylated glycans and/or the level of afucosylated glycans of that antibody composition. As further described herein, expressions, including but not limited to, Equations A-D and Equations 1-10, correlate FcγRII binding of an antibody composition with the % β-galactosylated glycan content and/or % of afucosylated glycan content of the antibody composition with statistical significance. Such expressions are useful in methods for predicting the level of FcγRII binding of an antibody composition based on the levels of these glycans. In various aspects, the predicted FcγRII binding level serves as a marker by which an antibody composition is identified as acceptable in terms of meeting a therapeutic threshold, and thus identifies ones which may be used in one or more downstream manufacturing process, or, alternatively, ones which are unacceptable and should not be carried forward in the manufacturing process. The presently disclosed correlations are further useful in identifying the glycoprofile of desired antibody compositions. With the correlations presented herein, and given a target FcγRII binding level, the glycoprofile (e.g., profile of β-galactosylated glycans, afucosylated glycans) of antibody compositions with the target FcγRII binding level are identified. With the identified profile of β-galactosylated glycans and afucosylated glycans of antibody compositions with the target FcγRII binding level, manufacturing processes, e.g., cell culturing, may be carried out to target that identified profile.

Accordingly, the present disclosure provides methods of determining product quality of an antibody composition. In various embodiments, the product quality is based on the level of FcγRII binding level of the antibody composition. In exemplary embodiments, the method comprises (a) determining the afucosylated glycan content and/or β-galactosylated glycan content of a sample of the antibody composition, (b) optionally, calculating a predicted FcγRII binding level based on the afucosylated glycan content and/or β-galactosylated glycan content determined in (a); and (c) determining the product quality of the antibody composition as acceptable when (i) the afucosylated glycan content and/or β-galactosylated glycan content is within a target range and/or (ii) the predicted FcγRII binding level is within a target range.

The present disclosure also provides methods of monitoring product quality of an antibody composition. In exemplary embodiments, the method comprises determining product quality of a first sample of an antibody composition obtained at a first timepoint in accordance with a presently disclosed method and determining product quality of a second sample of the antibody composition obtained at a second timepoint in accordance with a presently disclosed method, wherein the second timepoint is different from the first timepoint. In various aspects, the difference in level of afucosylated glycans and/or β-galactosylated glycans of the antibody composition between the first and second timepoints is informative of the difference in the level of FcγRII binding of the antibody composition.

The present disclosure additionally provides methods of producing an antibody composition. In exemplary embodiments, the method comprises determining the product quality of the antibody composition, wherein product quality of the antibody composition is determined in accordance with a method of the present disclosure, wherein the sample is a sample of in-process material, wherein, when the afucosylated glycan content and/or β-galactosylated glycan content determined in (a) is not within the target range, the method further comprises (d) modifying one or more conditions of the cell culture to obtain a modified cell culture and (e) determining the afucosylated glycan content and/or β-galactosylated glycan content of a sample of the antibody composition obtained from the modified cell culture, optionally, repeating (d) and (e) until the afucosylated glycan content and/or β-galactosylated glycan content is within the target range. In alternative or additional exemplary embodiments, the method comprises (a) determining the afucosylated glycan content and/or β-galactosylated glycan content of a sample of the antibody composition; (b) determining the FcγRII binding level of the antibody composition based on afucosylated glycan content and/or β-galactosylated glycan content determined in (a); and (c) selecting the antibody composition for downstream processing based on the level of FcγRII binding determined in (b).

Methods of modifying the level of FcγRII binding of an antibody composition are further provided. In exemplary embodiments, the method comprises (a) specifying a level of FcγRII; and (b) modifying the level of afucosylated glycans and/or β-galactosylated glycans of the antibody composition to achieve the specified level of FcγRII.

The present disclosure provides methods of determining the level of FcγRII binding of an antibody composition. In exemplary embodiments, the method comprises determining the level of afucosylated glycans and/or β-galactosylated glycans of the antibody composition. The present disclosure also provides a method of predicting the level of FcγRII binding of an antibody composition. In exemplary embodiments, the method comprises determining the level of afucosylated glycans and/or β-galactosylated glycans of the antibody composition. In various aspects, the level of afucosylated glycans and/or β-galactosylated glycans of the antibody composition is informative of the FcγRII binding of the antibody composition by virtue of the associations presented herein.

Methods of predicting in vivo efficacy and/or adverse effects of an antibody composition are provided by the present disclosure. In exemplary embodiments, the method comprises (a) determining the afucosylated glycan content and/or β-galactosylated glycan content of a sample of the antibody composition; and (b) predicting the antibody composition as causative of in vivo adverse effects based on the afucosylated glycan content and/or β-galactosylated glycan content determined in (a).

### BRIEF DESCRIPTION OF THE DRAWINGS

FIGS. 1A and 1B are illustrations of exemplary glycan structures.

FIG. 2A is a representative glycan map chromatogram (full scale view). FIG. 2B is a representative glycan map chromatogram (expanded scale view).

FIG. 3 is a general schematic of a part of the binding assay described in Examples 2 and 4.

FIG. 4A is an FcγRIIa binding leverage plot for β-galactosylated glycans. FIG. 4B is an FcγRIIa binding leverage plot for afucosylated glycans. FIG. 4C is an FcγRIIa binding leverage plot for HM glycans. FIG. 4D is a graph which plots actual FcγRIIa binding as a function of predicted FcγRIIa binding. FIG. 4E is a graph plotting FcγRIIa binding as a function of β-galactosylated glycans (%). The 95% confidence interval is shown as the shaded area. FIG. 4F is a graph plotting FcγRIIa binding as a function of afucosylated glycans (%). The 95% confidence interval is shown as the shaded area. FIG. 4G graph plotting FcγRIIa binding as a function of high mannose glycans (%). The 95% confidence interval is shown as the shaded area.

FIG. 5A is an FcγRIIb binding leverage plot for β-galactosylated glycans. FIG. 5B is an FcγRIIb binding leverage plot for afucosylated glycans. FIG. 5C is an FcγRIIb binding leverage plot for HM glycans. FIG. 5D is a graph which plots actual FcγRIIb binding as a function of predicted FcγRIIb binding. FIG. 5E is a graph plotting FcγRIIb binding as a function of β-galactosylated glycans (%). The 95% confidence interval is shown as the shaded area. FIG. 5F is a graph plotting FcγRIIb binding as a function of afucosylated glycans (%). The 95% confidence interval is shown as the shaded area. FIG. 5G is a graph plotting FcγRIIb binding as a function of high mannose glycans (%). The 95% confidence interval is shown as the shaded area.

FIG. 6 is an illustration of a presently disclosed correlation and exemplary applications thereof for drug substance manufacture and drug product release assay. In place of testing FcγRII binding or effector function of an in-process sample or sample of a lot, measure the % β-galactosylated glycans and % afucosylated glycans of the sample to determine whether the antibody composition should be selected for continued manufacturing or downstream processing or whether the lot should be released.

FIG. 7A is an FcγRIIa binding leverage plot for β-galactosylated glycans. FIG. 7B is an FcγRIIa binding leverage plot for afucosylated glycans. FIG. 7C is an FcγRIIa binding leverage plot for HM glycans. FIG. 7D is a graph which plots actual FcγRIIa binding as a function of predicted FcγRIIa binding. FIG. 7E is a graph plotting FcγRIIa binding as a function of β-galactosylated glycans (%). The 95% confidence interval is shown as the shaded area. FIG. 7F is a graph plotting FcγRIIa binding as a function of afucosylated glycans (%). The 95% confidence interval is shown as the shaded area. FIG. 7G graph plotting FcγRIIa binding as a function of high mannose glycans (%). The 95% confidence interval is shown as the shaded area.

FIG. 8A is an FcγRIIb binding leverage plot for β-galactosylated glycans. FIG. 8B is an FcγRIIb binding leverage plot for afucosylated glycans. FIG. 8C is an FcγRIIb binding leverage plot for HM glycans. FIG. 8D is a graph which plots actual FcγRIIb binding as a function of predicted FcγRIIb binding. FIG. 8E is a graph plotting FcγRIIb binding as a function of β-galactosylated glycans (%). The 95% confidence interval is shown as the shaded area. FIG. 8F is a graph plotting FcγRIIb binding as a function of afucosylated glycans (%). The 95% confidence interval is shown as the shaded area. FIG. 8G is a graph plotting FcγRIIb binding as a function of high mannose glycans (%). The 95% confidence interval is shown as the shaded area.

### DETAILED DESCRIPTION

### Glycosylation, Glycans, and Methods of Glycan Measurement

Many secreted proteins undergo post-translational glycosylation, a process by which sugar moieties (e.g., glycans, saccharides) are covalently attached to specific amino acids of a protein. In eukaryotic cells, two types of glycosylation reactions occur. (1) N-linked glycosylation, in which glycans are attached to the asparagine of the recognition sequence Asn-X-Thr/Ser, where “X” is any amino acid except proline, and (2) O-linked glycosylation in which glycans are attached to serine or threonine. Regardless of the glycosylation type (N-linked or O-linked), microheterogeneity of protein glycoforms exists due to the large range of glycan structures associated with each site (O or N).

All N-glycans have a common core sugar sequence: Manα1-6(Manα1-3)Manβ1-4GlcNAcβ1-4GlcNAcβ1-Asn-X-Ser/Thr (Man₃GlcNAc₂Asn) and are categorized into one of three types: (A) a high mannose (HM) or oligomannose (OM) type, which consists of two N-acetylglucosamine (GalNAc) moieties and a large number (e.g., 5, 6, 7, 8 or 9) of mannose (Man) residues (B) a complex type, which comprises more than two GlcNAc moieties and any number of other sugar types or (C) a hybrid type, which comprises a Man residue on one side of the branch and GlcNAc at the base of a complex branch.

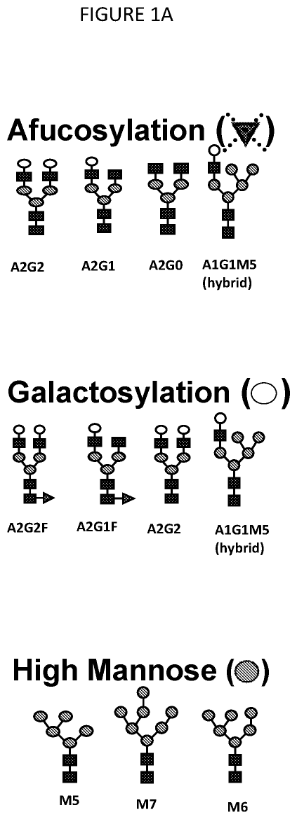

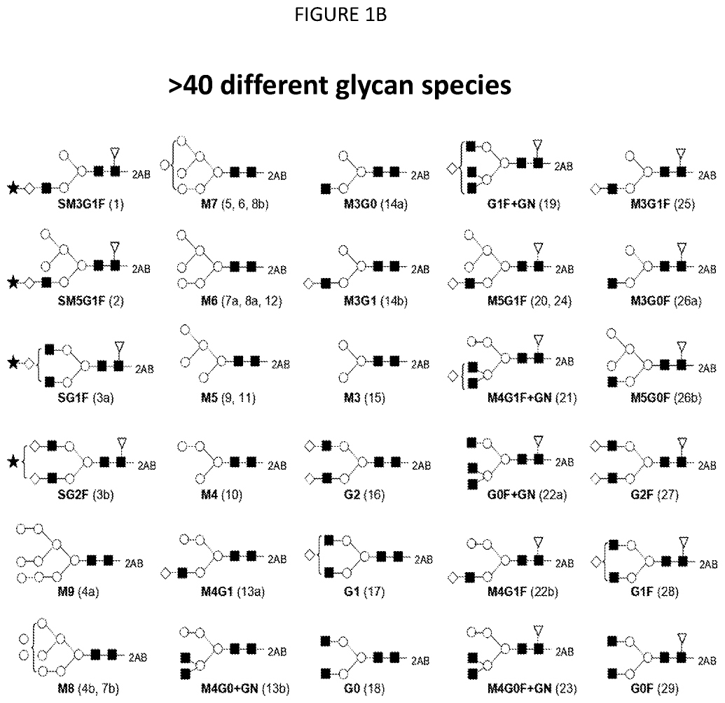

N-linked glycans typically comprise one or more monosaccharides of galactose (Gal), N-acetylgalactosamine (GalNAc), galactosamine (GaIN), glucose (Glc), N-acetylglucoasamine (GlcNAc), glucoasamine (GlcN), mannose (Man), N-Acetylmannosamine (ManNAc), Mannosamine (ManN), xylose (Xyl), N-Acetylneuraminic acid (Neu5Ac), N-Glycolylneuraminic acid (Neu5Gc), 2-keto-3-doxynononic acid (Kdn), fucose (Fuc), Glucuronic acid (GLcA), Iduronic acid (IdoA), Galacturonic acid (Gal A), mannuronic acid (Man A). Exemplary glycan structures illustrated with commonly used symbols for saccharides and their identity are shown in FIGS. 1A and 1B.

N-linked glycosylation begins in the endoplasmic reticulum (ER), where a complex set of reactions result in the attachment of a core glycan structure made essentially of two GlcNAc residues and three Man residues. The glycan complex formed in the ER is modified by action of enzymes in the Golgi apparatus. If the saccharide is relatively inaccessible to the enzymes, it typically stays in the original HM form. If enzymes can access the saccharide, then many of the Man residues are cleaved off and the saccharide is further modified, resulting in the complex type N-glycans structure. For example, mannosidase-1 located in the cis-Golgi, can cleave or hydrolyze a HM glycan, while fucosyltransferase FUT-8, located in the medial-Golgi, fucosylates the glycan (Hanrue Imai-Nishiya (2007), BMC Biotechnology, 7:84).

Accordingly, the sugar composition and the structural configuration of a glycan structure varies, depending on the glycosylation machinery in the ER and the Golgi apparatus, the accessibility of the machinery enzymes to the glycan structure, the order of action of each enzyme and the stage at which the protein is released from the glycosylation machinery, among other factors.

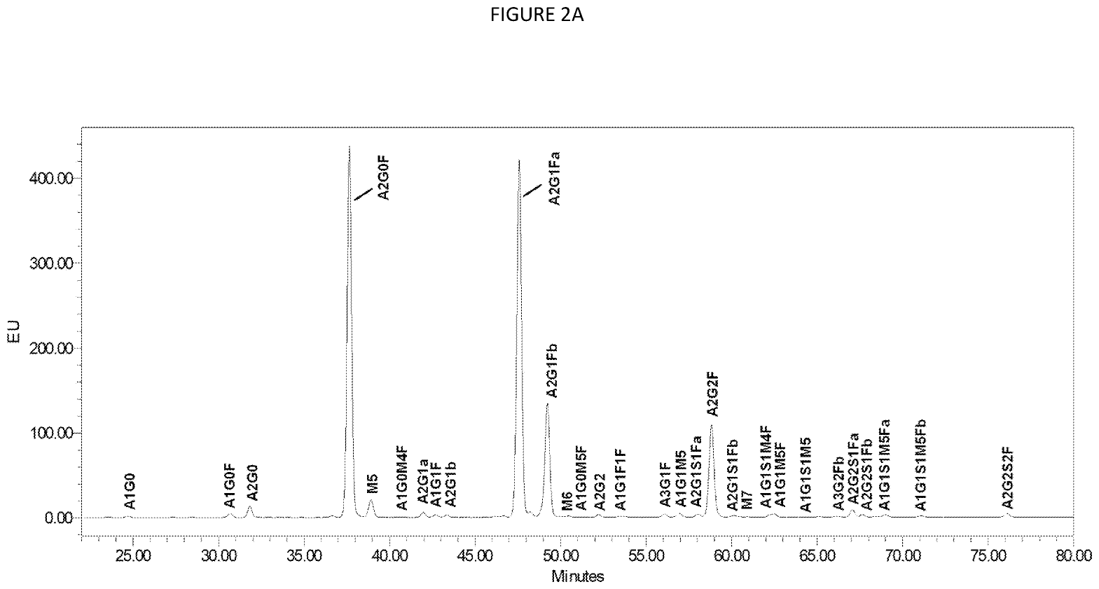

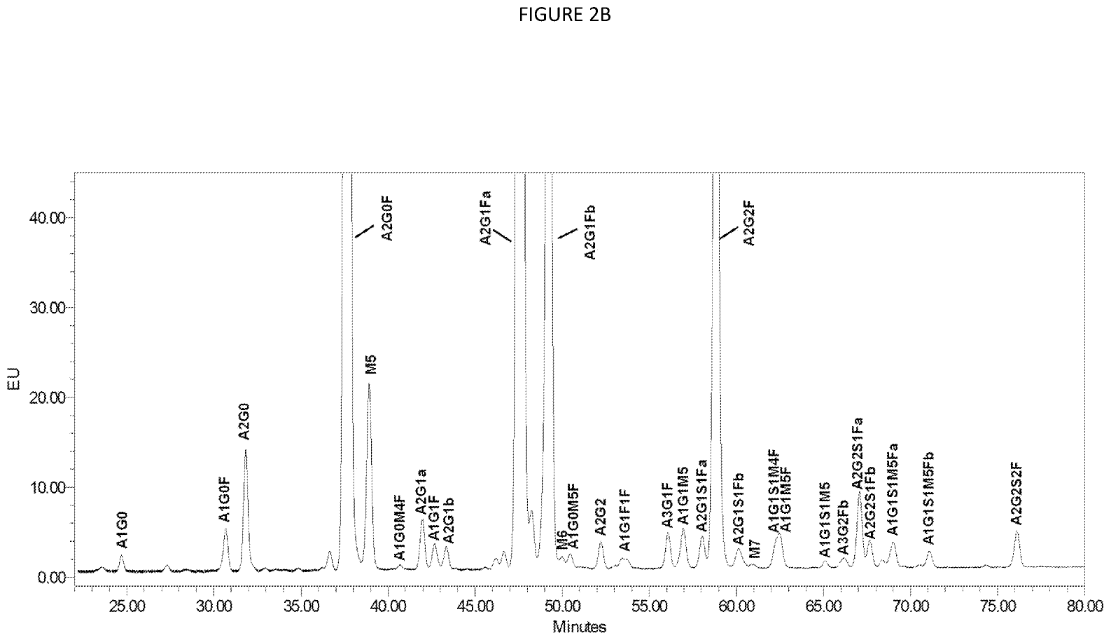

Various methods are known in the art for assessing glycans present in a glycoprotein-containing composition or for determining, detecting or measuring a glycoform profile (e.g., a glycoprofile) of a particular sample comprising glycoproteins. Suitable methods include, but are not limited to, positive ion MALDI-TOF analysis, negative ion MALDI-TOF analysis, weak anion exchange (WAX) chromatography, normal phase chromatography (NP-HPLC), exoglycosidase digestion, Bio-Gel P-4 chromatography, anion-exchange chromatography and one-dimensional n.m.r. spectroscopy, and combinations thereof. See, e.g., Mattu et al., JBC 273: 2260-2272 (1998); Field et al., Biochem J 299(Pt 1): 261-275 (1994); Yoo et al., MAbs 2(3): 320-334 (2010) Wuhrer M. et al., Journal of Chromatography B, 2005, Vol. 825, Issue 2, pages 124-133; Ruhaak L. R., Anal Bioanal Chem, 2010, Vol. 397:3457-3481 and Geoffrey, R. G. et. al. Analytical Biochemistry 1996, Vol. 240, pages 210-226. Also, Example 1 set forth herein describes a suitable method for assessing glycans present in a glycoprotein containing composition, e.g., an antibody composition. The method of Example 1 describes an assay in which glycans attached to glycosylated proteins of a composition, e.g., antibodies of an antibody composition, are enzymatically cleaved from the protein (e.g., antibody). The glycans are subsequently separated by Hydrophilic Interaction Liquid Chromatography (HILIC) and a chromatogram with several peaks is produced. Each peak of the chromatogram represents a mean distribution (amount) of a different glycan. Two views of an example HILIC chromatogram comprising peaks for different glycans are provided in FIGS. 2A and 2B. For these purposes, % Peak Area=Peak Area/Total Peak Area×100%, and % Total Peak Area=Sample Total Area/Total Area of the Standard×100%. Accordingly, the level of a particular glycan (or groups of glycans) is reported as a %. For example, if an antibody composition is characterized as having a Man6 level of 30%, it is meant that 30% of all glycans cleaved from the antibodies of the composition are Man6.

The present disclosure, including the correlations, associations, and equations presented herein, relates to afucosylated glycans and/or s-galactosylated glycans and/or high mannose glycans of an antibody composition. As used herein, the term “afucosylated glycan” or “AF glycan” refers to glycans which lack a core fucose, e.g., an a1,6-linked fucose on the GlcNAc residue involved in the amide bond with the Asn of the N-glycosylation site. Afucosylated glycans include, but are not limited to, A1G0, A2G0, A2G1a, A2G1b, A2G2, and A1G1M5. Additional afucosylated glycans include, e.g., A1G1a, G0[H3N4], G0[H4N4], G0[H5N4], FO-N[H3N3]. See, e.g., Reusch and Tejada, Glycobiology 25(12): 1325-1334 (2015). A level of afucosylated glycans, in various aspects, is obtained by summing the % of each afucosylated glycan species, e.g., summing % A1G0, the % A2G0, the % A2G1a, the % A2G1b, the % A2G2, the % A1G1M5, the % A1G1a, the % G0[H3N4], the % G0[H4N4], the % G0[H5N4], and the % FO-N[H3N3]. As used herein, the term “β-galactosylated glycan” is synonymous with “terminal galactose glycan” and refers to any glycan comprising one or two galactose molecules. A glycan comprising one galactose molecule is designated by “G1”, e.g., “G1a” or “G1b” in the glycan name, and a glycan comprising two galactose molecules is designated by “G2” in the glycan name. Accordingly, a β-galactosylated glycan in various aspects is a G1-galactosylated glycan, G1a-galactosylated glycan, G1b-galactosylated glycan, or a G2-galactosylated glycan. The β-galactosylated glycan in various aspects comprises a core fucose, e.g., A2G1F, A2G2F. Alternatively, the β-galactosylated glycan lacks a core fucose, e.g., A2G1 (including A2G1a and A2G1b) and A2G2 (or G1 and G2). In some embodiments, the galactosylated glycan is a hybrid glycan comprising a high mannose arm and a galactose-containing arm, as well as single-arm glycans exemplified by A1G1M5 and A1G1 respectively. It is noted that β-galactosylated glycans can lack core fucose (and thus represent a subset of afucosylated glycans), but β-galactosylated glycans have certain characteristics and may be referred to as a separate glycan group. Accordingly, unless explicitly stated otherwise, β-galactosylated glycan is understood to represent a separate characteristic and may be classified separately from, or as an additional characteristic of afucosylated glycans. A level of β-galactosylated glycans, in various aspects, is obtained by summing the % of each β-galactosylated glycan species, e.g., summing the % of each G1-galactosylated glycan species, each G1a-galactosylated glycan species, each G1b-galactosylated glycan species, and each G2-galactosylated glycan species. As used herein, the term “high mannose glycans” or “HM glycans” encompasses glycans comprising 5, 6, 7, 8, or 9 mannose residues, abbreviated as Man5, Man6, Man7, Man8, and Man9, respectively. A level of HM glycans, in various aspects, is obtained by summing the % Man5, the % Man6, the % Man7, the % Man8, and the % Man9.

In exemplary aspects, the level of glycans (e.g., the glycan content, optionally, expressed as a %, e.g., % AF glycans, % β-galactosylated glycans, % HM glycans) is determined (e.g., measured) by any of the various methods known in the art for assessing glycans present in a glycoprotein-containing composition or for determining, detecting or measuring a glycoform profile (e.g., a glycoprofile) of a particular sample comprising glycoproteins. In exemplary instances, the level of glycans (e.g., % AF glycans, % β-galactosylated glycans, % HM glycans) of an antibody composition is determined by measuring the level of such glycans in a sample of the antibody composition though a chromatography based method, e.g., HILIC, and the level of glycans is expressed as a %, as described herein. See, e.g., Example 1. In exemplary instances, the level of glycans of an antibody composition is expressed as a % of all glycans cleaved from the antibodies of the composition. In various aspects, the level of glycans (e.g., % AF glycans, % β-galactosylated glycans, % HM glycans) is determined (e.g., measured) by measuring the level of such glycans in a sample of the antibody composition. In exemplary instances, at least 5, at least 6, at least 7, at least 8, or at least 9 samples of an antibody composition are taken and the level of glycans (e.g., % AF glycans, % β-galactosylated glycans, % HM glycans) for each sample is determined (e.g., measured). In various aspects, the mean or average of the % AF glycans and/or % β-galactosylated glycans and/or % HM glycans is determined.

### FcγRII Binding

Fc receptors are receptors on the surfaces of B lymphocytes, follicular dendritic cells, natural killer (NK) cells, macrophages, neutrophils, eosinophils, basophils, platelets and mast cells that bind to the Fc region of an antibody. Fc receptors are grouped into different classes based on the type of antibody that they bind. For example, an Fcγ receptor is a receptor for the Fc region of an IgG antibody, an Fc-alpha receptor is a receptor for the Fc region of an IgA antibody, and an Fc-epsilon receptor is a receptor for the Fc region of an IgE antibody.

The term “FcγR” or “Fc-gamma receptor” refers to a protein belonging to the IgG superfamily involved in inducing phagocytosis of opsonized cells or microbes. See, e.g., Fridman W H. Fc receptors and immunoglobulin binding factors. FASEB Journal. 5 (12): 2684-90 (1991). Members of the Fc-gamma receptor family include: FcγRI (CD64), FcγRIIA (CD32), FcγRIIB (CD32), FcγRIIIA (CD16a), and FcγRIIIB (CD16b). The sequences of FcγRI, FcγRIIA, FcγRIIB, FcγRIIIA, and FcγRIIIB can be found in many sequence databases, for example, at the Uniprot database (www.uniprot.org) under accession numbers P12314 (FCGR1_HUMAN), P12318 (FCG2A_HUMAN), P31994 (FCG2B_HUMAN), P08637 (FCG3A_HUMAN), and P08637 (FCG3A_HUMAN), respectively.

The FcγRII family of human integral membrane receptor glycoproteins includes FcγRIIa, FcγRIIc and FcγRIIb. FcγRIIa and FcγRIIc have cellular functions which oppose the functions of FcγRIIb. FcγRIIa proteins are activating Fc receptors, whereas FcγRIIb is inhibitory and is considered as an immune checkpoint that modulates the action of activating-type Fc receptors and the antigen receptor of B cells. FcγRIIc is similar to FcγRIIa and is considered as an activating Fc receptor. FcγRIIa is expressed on granulocytes, monocytes and monocyte-derived cells such as macrophages and dendritic cells (DCs). Engagement of FcγRIIa by IgG crosslinking can initiate a variety of effector functions, including, for instance, phagocytosis, activation of neutrophil and other myeloid effector cells for killing of IgG-opsonized target cells, activation of granulocytes to release inflammatory mediators, T cell proliferation and T cell-mediated cytokine secretion, and platelet activation, adhesion and aggregation following vessel injury. The structure and functions of the FcγRII proteins are reviewed in Anania et al., Front. Immunol. 10: 464 (2019); accessible on the world wide web at doi.org/10.3389/fimmu.2019.00464.

The present disclosure, including the correlations, associations, and equations presented herein, relates to the level of FcγRII binding of an antibody composition. While methods of measuring the FcγRII binding level of an antibody composition are known in the art, exemplary methods of which are described herein (see, e.g., Example 2 and 4), the data presented herein support that the level of FcγRII binding of an antibody composition may be predicted by the glycoprofile of the antibody composition. In exemplary instances, the % afucosylated glycans and/or the % β-galactosylated glycans and/or the % HM glycans of an antibody composition may be used to calculate or predict the level of FcγRII binding for the antibody composition. Also, given that antibody effector functions are induced upon binding of an antibody Fc domain with an FcγRII, the level of FcγRII binding of an antibody composition, in various instances, serves as a surrogate for effector function, such that the % afucosylated glycans and/or the % β-galactosylated glycans and/or the % HM glycans of an antibody composition may be used to calculate or predict the level of effector function of the antibody composition, wherein the effector function is activated upon FcγRII binding. In exemplary aspects, the present disclosure relates the % afucosylated glycans and/or the % β-galactosylated glycans and/or the % HM glycans of an antibody composition to the level of FcγRIIa binding. In alternative or additional aspects, the present disclosure relates the % afucosylated glycans and/or the % β-galactosylated glycans and/or the % HM glycans of an antibody composition to the level of FcγRIIb binding.

The presently disclosed relationships connecting the % afucosylated glycans and/or the % β-galactosylated glycans and/or the % HM glycans of an antibody composition to the level of FcγRII binding in various instances are useful for designing process control measures to ensure that the desired FcγRII binding activity can be delivered consistently and at the level intended. The correlations may be exploited to assure consistent clinical performance, for achieving functional similarity of biosimilar candidates, and to predict potential adverse in vivo effects of therapeutic antibody treatment.

In various aspects, based on the present disclosures, the FcγRII binding level may be calculated based on the % afucosylated glycans and/or the % β-galactosylated glycans and/or the % HM glycans of an antibody composition. In various aspects, the % afucosylated glycans and/or the % β-galactosylated glycans and/or the % HM glycans of the antibody composition is/are measured amounts based on a sample of the antibody composition. In various instances, the measured % afucosylated glycans and/or the measured % β-galactosylated glycans and/or the measured % HM glycans are measured by a method including but not limited to HILIC. In various instances, the measured % afucosylated glycans and/or the measured % β-galactosylated glycans and/or the measured % HM glycans are measured by a method including but not limited to the method described in Example 1.

In various aspects, based on the present disclosures, the % afucosylated glycans and/or the % β-galactosylated glycans and/or the % HM glycans may be calculated based on a known or predetermined or pre-selected or target FcγRII binding level. In various instances, a target FcγRII binding level or target range of FcγRII binding levels is known, given the particular antibody of the antibody composition being produced. For example, the antibody may comprise the same amino acid sequence as a reference antibody (or an amino acid sequence at least 95%, 97%, or 99% identical to that of the reference antibody), and the target FcγRII binding level or a range thereof is known for the reference antibody. In exemplary aspects, the target % afucosylated glycans and/or the target % β-galactosylated glycans and/or the target % HM glycans is/are calculated based on a first model which correlates the % afucosylated glycans and/or the % β-galactosylated glycans and/or the % HM glycans with FcγRII binding level. In various instances, the first model is a linear regression model. In various aspects, the first model which correlates FcγRII binding level with the % afucosylated glycans and/or the % β-galactosylated glycans and/or the % HM glycans is statistically significant as demonstrated by its low p-value. In various aspects, the p-value is less than 0.05. In various instances, the p-value is less than 0.01 or less than 0.001. In various instances, the p-value is less than 0.0001.

In exemplary aspects, the β-galactosylated glycan content of an antibody composition positively correlates with the FcγRII binding level. In various aspects, higher levels of β-galactosylated glycan content correlate with higher FcγRII binding levels and lower levels of β-galactosylated glycan content correlate with lower FcγRII binding levels. In exemplary aspects, the afucosylated glycan content of an antibody composition negatively correlates with the FcγRII binding level. In various aspects, higher levels of afucosylated glycan content correlate with lower FcγRII binding levels and lower levels of afucosylated glycan content correlate with higher FcγRII binding levels. In exemplary aspects, high mannose glycan content of an antibody composition correlates with the FcγRII binding level. In exemplary instances, the correlation is a negative correlation. In various aspects, higher levels of HM glycan content correlate with lower FcγRII binding levels and lower levels of HM glycan content correlate with higher FcγRII binding levels.

In exemplary aspects, the FcγRII binding level is a level of FcγRIIa binding. In exemplary instances, a FcγRIIa binding level is calculated based on a determined or measured β-galactosylated glycan content (e.g., % β-galactosylated glycans). In various aspects, the FcγRII binding level is calculated according to Equation A:

$$Fc γ RII binding level = {m}^{*} \% BG + y , \qquad [ Equation A ]$$

wherein m is about 0.535 to about 1.091, y is about 72.58 to about 85.78, and % BG is the % β-galactosylated glycan content determined in (a).

In exemplary instances, m of Equation A is 0.813 and/or y of Equation A is 79.18. In alternative exemplary instances, m of Equation A is 0.778 and/or y of Equation A is 81.76.

In exemplary instances, a FcγRII binding level is calculated based on a determined or measured afucosylated glycan content (e.g., % afucosylated glycan). The FcγRII binding level, in various instances, is calculated according to Equation B:

$$Fc γ RII binding level = {m}^{*} \% AF + y , \qquad [ Equation B ]$$

wherein m is about −13.73 to about −7.54, y is about 108.8 to about 119.1, and % AF is the % afucosylated glycan content.

In various aspects, m of Equation B is −10.63 and/or y of Equation B is 114. In alternative exemplary instances, m of Equation B is −9.53 and/or y of Equation B is 114.

In exemplary aspects, FcγRII binding level is based on a determined or measured afucosylated glycan content (e.g., % afucosylated glycan) and a determined or measured β-galactosylated glycan content (e.g., % β-galactosylated glycan).

In exemplary instances, the FcγRII binding level is a level within the 95% confidence interval of a line of Equation 3:

$$Fc γ RII binding level = {0.576}^{*} \% BG + {( - 4.978 )}^{*} \% AF + 98.877 , \qquad [ Equation 3 ]$$

wherein % BG is the % β-galactosylated glycan content and % AF is the % afucosylated glycan content.

In exemplary aspects, the FcγRII binding level is a level of FcγRIIb binding. In exemplary instances, a FcγRIIb binding level is calculated based on a determined or measured β-galactosylated glycan content. (e.g., % β-galactosylated glycans). In various aspects, the FcγRII binding level is calculated according to Equation C:

$$Fc γ RII binding level = {m}^{*} \% BG + y , \qquad [ Equation C ]$$

wherein m is about 0.3260 to about 0.9697, y is about 77.72 to about 92.99, and % BG is the % β-galactosylated glycan content.

In various instances, m of Equation C is 0.648 and/or y of Equation C is 85.36. In alternative exemplary instances, m of Equation C is 0.644 and/or y of Equation C is 86.34.

In exemplary instances, a FcγRIIb binding level is calculated based on a determined or measured afucosylated glycan content (e.g., % afucosylated glycan). The FcγRII binding level is in various instances calculated according to Equation D:

$$Fc γ RII binding level = {m}^{*} \% AF + y , \qquad [ Equation D ]$$

wherein m is about −12.02 to about −6.247, y is about 109.3 to about 118.9, and % AF is the % afucosylated glycan content.

In various aspects, m of Equation D is about −9.132 and/or y of Equation D is about 114. In alternative exemplary instances, m of Equation D is −7.102 and/or y of Equation D is 111.9.

In various aspects, a FcγRIIb binding level is calculated based on a determined or measured afucosylated glycan content (e.g., % afucosylated glycan) and a determined or measured β-galactosylated glycan content (e.g., % β-galactosylated glycan). In exemplary instances, the FcγRII binding level is a level within the 95% confidence interval of a line of Equation 4:

$$Fc γ RII binding level = {0.461}^{*} \% BG + {( - 4.429 )}^{*} \% AF + 105.731 , \qquad [ Equation 4 ]$$

wherein % BG is the % β-galactosylated glycan content and % AF is the % afucosylated glycan content.

In exemplary instances, a FcγRII binding level is calculated based on a determined or measured high mannose (HM) glycan content (e.g., % HM glycan). In various aspects, a FcγRII binding level is calculated based on a determined or measured afucosylated glycan content (e.g., % afucosylated glycan), a determined or measured β-galactosylated glycan content (e.g., % β-galactosylated glycan), and a determined or measured HM glycan content (% HM glycan). In exemplary aspects, the FcγRIIa binding level of an antibody composition is a level within the 95% confidence interval of a line of Equation 5:

$$Fc γ RII binding = {0.576}^{*} \% BG + {( - 4.978 )}^{*} \% AF + 98.877 + {( - 1.343 )}^{*} \% HM , \qquad [ Equation 5 ]$$

wherein % BG is the % β-galactosylated glycan content, % AF is the % afucosylated glycan content, and % HM is the % high mannose glycan content.

In exemplary aspects, the FcγRIIa binding level of an antibody composition is a level within the 95% confidence interval of a line of Equation 9:

$$Fc γ RII binding = {0.545}^{*} \% BG + {( ∼ 4.466 )}^{*} \% AF + 102.7 + {( - 2.036 )}^{*} \% HM , \qquad [ Equation 9 ]$$

wherein % BG is the % β-galactosylated glycan content, % AF is the % afucosylated glycan content, and % HM is the % high mannose glycan content.

In exemplary aspects, the FcγRIIb binding level of an antibody composition is a level within the 95% confidence interval of a line of Equation 6:

$$Fc γ RII binding = {0.461}^{*} \% BG + {( - 4.429 )}^{*} \% AF + 105.731 + {( - 1.883 )}^{*} \% HM , \qquad [ Equation 6 ]$$

wherein % BG is the % β-galactosylated glycan content, % AF is the % afucosylated glycan content, and % HM is the % high mannose glycan content.

In exemplary aspects, the FcγRIIb binding level of an antibody composition is a level within the 95% confidence interval of a line of Equation 10:

$$Fc γ RII binding = {0.59}^{*} \% BG + {( ∼ 2.04 )}^{*} \% AF + 99.2 + {( - 1.91 )}^{*} \% HM , \qquad [ Equation 10 ]$$

wherein % BG is the % β-galactosylated glycan content, % AF is the % afucosylated glycan content, and % HM is the % high mannose glycan content.
   Methods of Determining and/or Monitoring Product Quality

Based on the present disclosure, product quality of an antibody composition may be determined and/or monitored. Accordingly, the present disclosure provides methods of determining product quality of an antibody composition, wherein the product quality of the antibody composition is based on the FcγRII binding level of the antibody composition. In exemplary embodiments, the method comprises (a) determining the afucosylated glycan content and/or the β-galactosylated glycan content of a sample of an antibody composition; (b) optionally, calculating a FcγRII binding level based on the afucosylated glycan content and/or β-galactosylated glycan content as determined in (a); and (c) determining the product quality of the antibody composition as acceptable when (i) the afucosylated glycan content and/or β-galactosylated glycan content is within a target range and/or (ii) the FcγRII binding level is within a target range.

In various aspects, the target range of FcγRII binding levels, the target range of the afucosylated glycan content and/or the target range of the β-galactose glycan content is based on the FcγRII binding levels, the afucosylated glycan content, and/or the β-galactose glycan content of a reference antibody. In various instances, the reference antibody comprises a chimeric constant region. In exemplary instances, the chimeric constant region of the reference antibody comprises a portion of an IgG2 constant region and a portion of an IgG4 constant region. In various aspects, the chimeric constant region comprises CH1 and/or a hinge of an IgG2 and/or CH2-CH3 of an IgG4. In exemplary instances, the chimeric constant region comprises a chimeric constant region of SEQ ID NO: 15. Optionally, the reference antibody is eculizumab.

In exemplary aspects, the FcγRII binding level is a level of FcγRIIa binding. In exemplary instances, a FcγRIIa binding level is calculated based on a determined or measured β-galactosylated glycan content (e.g., % β-galactosylated glycans). In various aspects, the FcγRII binding level is calculated according to Equation A:

$$Fc γ RII binding level = {m}^{*} \% BG + y , \qquad [ Equation A ]$$

wherein m is about 0.535 to about 1.091, y is about 72.58 to about 85.78, and % BG is the % β-galactosylated glycan content determined in (a).

In exemplary instances, m of Equation A is 0.813 and/or y of Equation A is 79.18. In alternative exemplary instances, m of Equation A is 0.778 and/or y of Equation A is 81.76.

In exemplary instances, a FcγRII binding level is calculated based on a determined or measured afucosylated glycan content (e.g., % afucosylated glycan). The FcγRII binding level, in various instances, is calculated according to Equation B:

$$Fc γ RII binding level = m * \% AF + y , \qquad [ Equation B ]$$

wherein m is about −13.73 to about −7.54, y is about 108.8 to about 119.1, and % AF is the % afucosylated glycan content.

In various aspects, m of Equation B is −10.63 and/or y of Equation B is 114. In alternative exemplary instances, m of Equation B is −9.53 and/or y of Equation B is 114.

In exemplary aspects, FcγRII binding level is based on a determined or measured afucosylated glycan content (e.g., % afucosylated glycan) and a determined or measured β-galactosylated glycan content (e.g., % β-galactosylated glycan).

In exemplary instances, the FcγRII binding level is a level within the 95% confidence interval of a line of Equation 3:

$$Fc γ RII binding = 0.576 * \% BG + ( - 4.978 ) * \% AF + 98.877 , \qquad [ Equation 3 ]$$

wherein % BG is the % β-galactosylated glycan content and % AF is the % afucosylated glycan content.

In exemplary aspects, the FcγRII binding level is a level of FcγRIIb binding. In exemplary instances, a FcγRIIb binding level is calculated based on a determined or measured β-galactosylated glycan content. (e.g., % β-galactosylated glycans). In various aspects, the FcγRII binding level is calculated according to Equation C:

$$Fc γ RII binding level = m * \% BG + y , \qquad [ Equation C ]$$

wherein m is about 0.3260 to about 0.9697, y is about 77.72 to about 92.99, and % BG is the % β-galactosylated glycan content.

In various instances, m of Equation C is 0.648 and/or y of Equation C is 85.36. In alternative exemplary instances, m of Equation C is 0.644 and/or y of Equation C is 86.34.

In exemplary instances, a FcγRIIb binding level is calculated based on a determined or measured afucosylated glycan content (e.g., % afucosylated glycan). The FcγRII binding level is in various instances calculated according to Equation D:

$$Fc γ RII binding level = m * \% AF + y , \qquad [ Equation D ]$$

wherein m is about −12.02 to about −6.247, y is about 109.3 to about 118.9, and % AF is the % afucosylated glycan content.

In various aspects, m of Equation D is about −9.132 and/or y of Equation D is about 114. In alternative exemplary instances, m of Equation D is −7.102 and/or y of Equation D is 111.9.

In various aspects, a FcγRIIb binding level is calculated based on a determined or measured afucosylated glycan content (e.g., % afucosylated glycan) and a determined or measured β-galactosylated glycan content (e.g., % β-galactosylated glycan). In exemplary instances, the FcγRII binding level is a level within the 95% confidence interval of a line of Equation 4:

$$Fc γ RII binding = 0.461 * \% BG + ( - 4.429 ) \% AF + 105.731 , \qquad [ Equation 4 ]$$

wherein % BG is the % β-galactosylated glycan content and % AF is the % afucosylated glycan content.

In exemplary instances, a FcγRII binding level is calculated based on a determined or measured high mannose (HM) glycan content (e.g., % HM glycan). In various aspects, a FcγRII binding level is calculated based on a determined or measured afucosylated glycan content (e.g., % afucosylated glycan), a determined or measured β-galactosylated glycan content (e.g., % β-galactosylated glycan), and a determined or measured HM glycan content (% HM glycan). In exemplary aspects, the FcγRIIa binding level of an antibody composition is a level within the 95% confidence interval of a line of Equation 5:

$$Fc γ RII binding = 0.576 * \% BG + ( - 4.978 ) * \% AF + 98.877 + ( - 1.343 ) * \% HM , \qquad [ Equation 5 ]$$

wherein % BG is the % β-galactosylated glycan content, % AF is the % afucosylated glycan content, and % HM is the % high mannose glycan content.

In exemplary aspects, the FcγRIIa binding level of an antibody composition is a level within the 95% confidence interval of a line of Equation 9:

$$Fc γ RII binding = 0.545 * \% BG + ( - 4.466 ) * \% AF + 102.7 + ( - 2.036 ) * \% HM , \qquad [ Equation 9 ]$$

wherein % BG is the % β-galactosylated glycan content, % AF is the % afucosylated glycan content, and % HM is the % high mannose glycan content.

In exemplary aspects, the FcγRIIb binding level of an antibody composition is a level within the 95% confidence interval of a line of Equation 6:

$$Fc γ RII binding = 0.461 ⋆ \% BG + ( - 4.429 ) * \% AF + 105.7 \; 31 + ( - 1.883 ) * \% HM , \qquad [ Equation 6 ]$$

wherein % BG is the % β-galactosylated glycan content, % AF is the % afucosylated glycan content, and % HM is the % high mannose glycan content.

In exemplary aspects, the FcγRIIb binding level of an antibody composition is a level within the 95% confidence interval of a line of Equation 10:

$$Fc γ RII binding = 0.59 * \% BG + ( - 2.04 ) * \% AF + 99.2 + ( - 1.91 ) * \% HM , \qquad [ Equation 10 ]$$

wherein % BG is the % β-galactosylated glycan content, % AF is the % afucosylated glycan content, and % HM is the % high mannose glycan content.

In exemplary aspects, the method is a quality control (QC) assay. In exemplary aspects, the method is an in-process QC assay. In various aspects, the sample is a sample of in-process material. In various instances, the AF glycan content and/or the β-galactosylated glycan content is determined pre-harvest or post-harvest. In exemplary instances, the AF glycan content and/or the β-galactosylated glycan content is determined after chromatography. Optionally, the chromatography comprises a capture chromatography, intermediate chromatography, and/or polish chromatography. In some aspects, the AF glycan content and/or the β-galactosylated glycan content is determined after a virus inactivation and neutralization, virus filtration, or a buffer exchange. The method in various instances is a lot release assay. The sample in some aspects is a sample of a manufacturing lot.

In various aspects, the method further comprises selecting the antibody composition for downstream processing, when (i) the afucosylated glycan content and/or β-galactosylated glycan content is within a target range and/or (ii) the FcγRII binding level is within a target range. When the AF glycan content and/or the β-galactosylated glycan content determined in (a) is not within the target range, one or more conditions of the cell culture are modified to obtain a modified cell culture, in various aspects. The method, in some aspects, further comprises determining the afucosylated glycan content and/or β-galactosylated glycan content of a sample of the antibody composition obtained after one or more conditions of the cell culture are modified, e.g., determining the afucosylated glycan content and/or β-galactosylated glycan content of a sample of the antibody composition of the modified cell culture. In various aspects, when the afucosylated glycan content and/or β-galactosylated glycan content determined in (a) is not within the target range, the method further comprises (d) modifying one or more conditions of the cell culture to obtain a modified cell culture and (e) determining the afucosylated glycan content and/or β-galactosylated glycan content of a sample of the antibody composition obtained from the modified cell culture. In exemplary aspects, when the afucosylated glycan content and/or β-galactosylated glycan content determined in (a) is not within the target range, the method further comprises (d) and (e) until the afucosylated glycan content and/or β-galactosylated glycan content determined in (d) is within the target range.

In exemplary instances, an assay which directly measures FcγRII binding of the antibody composition is carried out on the antibody composition only when the afucosylated glycan content and/or β-galactosylated glycan content determined in (a) is not within the target range, e.g., outside the target range. Assays which directly measure FcγRII binding activity include for example the assay described in Example 2 or Example 4. In exemplary instances, an assay which directly measures FcγRII binding of the antibody composition is not carried out on the antibody composition. In various aspects, determining the afucosylated glycan content and/or β-galactosylated glycan content is the only step required to determine the product quality of the antibody composition. Without being bound to theory, the statistically significant correlations described herein allow for afucosylated glycan content and/or β-galactosylated glycan content to indicate FcγRII binding level such that assays that directly measure FcγRII binding level are not needed. Accordingly, direct measurement of the FcγRII binding level of the antibody composition is not needed and thus not carried out in various aspects of the presently disclosed methods.

In various aspects, the method determines the product quality in terms of the FcγRII binding level criterion. In various aspects, the FcγRII binding level criterion is one of the acceptance criteria for the antibody composition. The presently disclosed methods in various aspects are purposed to assure that batches of drug products meet each appropriate specification and appropriate statistical quality control criteria as a condition for their approval and release, for example approval and release pursuant to 21 CFR 211.165 in the United States. In various aspects, the presently disclosed methods of determining product quality meet the statistical quality control criteria which includes appropriate acceptance levels and/or appropriate rejection levels. Terminology, including, but not limited to “acceptance criteria”, “lot” and “in-process” accord with their meaning as defined in 21 Code of Federal Regulations (CFR) Section 210.3.

The present disclosure also provides methods of monitoring product quality of an antibody composition, wherein the FcγRII binding level of the antibody composition is a criterion upon which product quality of the antibody composition is based. In exemplary embodiments, the method comprises determining product quality of an antibody composition in accordance with a method of the present disclosures, with a first sample obtained at a first timepoint and with a second sample taken at a second timepoint which is different from the first timepoint. In various instances, each of the first sample and second sample is a sample of in-process material. In various aspects, the first sample is a sample of in-process material and the second sample is a sample of a manufacturing lot. Optionally, the first sample is a sample obtained before one or more conditions of the cell culture are modified and the second sample is a sample obtained after the one or more conditions of the cell culture are modified. In exemplary instances, the afucosylated glycan content and/or β-galactosylated glycan content is determined for each of the first sample and second sample. Additional samples may be obtained for purposes of determining product quality of the antibody composition and for determining afucosylated glycan content and/or β-galactosylated glycan content. Product quality of the antibody composition depends on whether the afucosylated glycan content and/or β-galactosylated glycan content is within a target range. In exemplary aspects, the target range of afucosylated glycan content and/or β-galactosylated glycan content is based on a reference antibody. In various aspects, the target range of FcγRII binding levels, the target range of the afucosylated glycan content and/or the target range of the β-galactose glycan content is based on the FcγRII binding levels, the afucosylated glycan content, and/or the β-galactose glycan content of a reference antibody. In various instances, the reference antibody comprises a chimeric constant region. In exemplary instances, the chimeric constant region of the reference antibody comprises a portion of an IgG2 constant region and a portion of an IgG4 constant region. In various aspects, the chimeric constant region comprises CH1 and/or a hinge of an IgG2 and/or CH2-CH3 of an IgG4. In exemplary instances, the chimeric constant region comprises a chimeric constant region of SEQ ID NO: 15. Optionally, the reference antibody is eculizumab.

### Methods of Producing Antibody Compositions

The present disclosure provides methods of producing an antibody composition. In exemplary embodiments, the method comprises determining product quality of the antibody composition wherein product quality of the antibody composition is determined in accordance with a method of the present disclosures. Optionally, the method comprises determining the afucosylated glycan content and/or β-galactosylated glycan content of a sample of an antibody composition and the sample is a sample of in-process material. In various instances, the method comprises determining the product quality of the antibody composition as acceptable and/or achieving the FcγRII binding level criterion when the afucosylated glycan content and/or β-galactosylated glycan content determined in (a) is within a target range, as defined herein. In exemplary aspects, the target range of afucosylated glycan content and/or β-galactosylated glycan content is based on the target range of FcγRII binding levels for a reference antibody. In various aspects, when the afucosylated glycan content and/or β-galactosylated glycan content determined in (a) is not within the target range, the method further comprises (iii) modifying one or more conditions of the cell culture to obtain a modified cell culture and (d) determining the afucosylated glycan content and/or β-galactosylated glycan content of a sample of the antibody composition obtained from the modified cell culture, optionally, repeating (iii) and (e) until the afucosylated glycan content and/or β-galactosylated glycan content is within the target range. In various instances, the sample is a sample of a cell culture comprising cells expressing an antibody of the antibody composition. In various instances, one or more conditions of the cell culture are modified to modify the afucosylated glycan content and/or β-galactosylated glycan content. In various instances, a host cell or clone is selected to obtain the modified afucosylated glycan content and/or β-galactosylated glycan content. In various aspects, the method comprises modifying the AF glycan content. In exemplary aspects, one or more conditions of the cell culture are modified to modify the AF glycan content of the antibody composition. In exemplary aspects, the one or more conditions primarily modify the AF glycan content. In various instances, the one or more conditions modify the AF glycan content and does not modify the β-galactosylated glycan content. In exemplary aspects, the method comprises modifying the β-galactosylated glycan content. Optionally, one or more conditions of the cell culture are modified to modify the β-galactosylated glycan content of the antibody composition. In some instances, the one or more conditions primarily modify the β-galactosylated glycan content. In some aspects, the one or more conditions modify the β-galactosylated glycan content and does not modify the AF glycan content. In various instances, the method comprises repeating the modifying of the afucosylated (AF) glycan content and/or repeating the modifying of the β-galactosylated glycan, until both of the afucosylated glycan content and β-galactosylated glycan content are within a target range. Ultimately, the method comprises modifying the afucosylated (AF) glycan content and/or modifying of the β-galactosylated glycan, until the FcγRII binding (as calculated or predicted) is within a target range. In various aspects, one or more conditions of the cell culture are modified to primarily change the HM glycan content to achieve the target range of FcγRII binding and/or one or more conditions of the cell culture are modified to primarily change the β-galactosylated glycan content to achieve the target range of FcγRII binding.

In exemplary aspects, the target ranges are the target ranges of a reference antibody. For example, if the target range of FcγRII binding levels of a reference antibody is known, the target level of the afucosylated glycan content and/or β-galactosylated glycan content may be calculated according to the correlations set forth herein. Alternatively, if the target range of afucosylated glycan content of a reference antibody is known and/or a target range of β-galactosylated glycan content of a reference antibody is known, the target range of FcγRII binding levels of a reference antibody may be calculated.

In exemplary aspects, the FcγRII binding level is a level of FcγRIIa binding. In exemplary instances, a FcγRIIa binding level is calculated based on a determined or measured β-galactosylated glycan content (e.g., % β-galactosylated glycans). In various aspects, the FcγRII binding level is calculated according to Equation A:

$$Fc γ RII binding level = m * \% BG + y , \qquad [ Equation A ]$$

wherein m is about 0.535 to about 1.091, y is about 72.58 to about 85.78, and % BG is the % β-galactosylated glycan content determined in (a).

In exemplary instances, m of Equation A is 0.813 and/or y of Equation A is 79.18. In alternative exemplary instances, m of Equation A is 0.778 and/or y of Equation A is 81.76.

In exemplary instances, a FcγRII binding level is calculated based on a determined or measured afucosylated glycan content (e.g., % afucosylated glycan). The FcγRII binding level, in various instances, is calculated according to Equation B:

$$Fc γ RII binding level = m * \% AF + y , \qquad [ Equation B ]$$

wherein m is about −13.73 to about −7.54, y is about 108.8 to about 119.1, and % AF is the % afucosylated glycan content.

In various aspects, m of Equation B is −10.63 and/or y of Equation B is 114. In alternative exemplary instances, m of Equation B is −9.53 and/or y of Equation B is 114.

In exemplary aspects, FcγRII binding level is based on a determined or measured afucosylated glycan content (e.g., % afucosylated glycan) and a determined or measured β-galactosylated glycan content (e.g., % β-galactosylated glycan).

In exemplary instances, the FcγRII binding level is a level within the 95% confidence interval of a line of Equation 3:

$$Fc γ RII binding = 0.576 * \% BG + ( - 4. 9 \; 78 ) * \% AF + 98.877 , \qquad [ Equation 3 ]$$

wherein % BG is the % β-galactosylated glycan content and % AF is the % afucosylated glycan content.

In exemplary aspects, the FcγRII binding level is a level of FcγRIIb binding. In exemplary instances, a FcγRIIb binding level is calculated based on a determined or measured β-galactosylated glycan content. (e.g., % β-galactosylated glycans). In various aspects, the FcγRII binding level is calculated according to Equation C:

$$Fc γ RII binding level = m * \% BG + y , \qquad [ Equation C ]$$

wherein m is about 0.3260 to about 0.9697, y is about 77.72 to about 92.99, and % BG is the % β-galactosylated glycan content.

In various instances, m of Equation C is 0.648 and/or y of Equation C is 85.36. In alternative exemplary instances, m of Equation C is 0.644 and/or y of Equation C is 86.34.

In exemplary instances, a FcγRIIb binding level is calculated based on a determined or measured afucosylated glycan content (e.g., % afucosylated glycan). The FcγRII binding level is in various instances calculated according to Equation D:

$$Fc γ RII binding level = m * \% AF + y , \qquad [ Equation D ]$$

wherein m is about −12.02 to about −6.247, y is about 109.3 to about 118.9, and % AF is the % afucosylated glycan content.

In various aspects, m of Equation D is about −9.132 and/or y of Equation D is about 114. In alternative exemplary instances, m of Equation D is −7.102 and/or y of Equation D is 111.9.

In various aspects, a FcγRIIb binding level is calculated based on a determined or measured afucosylated glycan content (e.g., % afucosylated glycan) and a determined or measured β-galactosylated glycan content (e.g., % β-galactosylated glycan). In exemplary instances, the FcγRII binding level is a level within the 95% confidence interval of a line of Equation 4:

$$Fc γ RII binding = 0.461 * \% BG + ( - 4.429 ) \% AF + 105.731 , \qquad [ Equation 4 ]$$

wherein % BG is the % β-galactosylated glycan content and % AF is the % afucosylated glycan content.

In exemplary instances, a FcγRII binding level is calculated based on a determined or measured high mannose (HM) glycan content (e.g., % HM glycan). In various aspects, a FcγRII binding level is calculated based on a determined or measured afucosylated glycan content (e.g., % afucosylated glycan), a determined or measured β-galactosylated glycan content (e.g., % β-galactosylated glycan), and a determined or measured HM glycan content (% HM glycan). In exemplary aspects, the FcγRIIa binding level of an antibody composition is a level within the 95% confidence interval of a line of Equation 5:

$$[ Equation 5 ]$$

$$FcyRII binding = 0.576 * \% BG + ( - 4.978 ) * \% AF + 98.877 + ( - 1.343 ) * \% HM ,$$

wherein % BG is the % β-galactosylated glycan content, % AF is the % afucosylated glycan content, and % HM is the % high mannose glycan content.

In exemplary aspects, the FcγRIIa binding level of an antibody composition is a level within the 95% confidence interval of a line of Equation 9:

$$[ Equation 9 ]$$

$$FcyRII binding = 0.545 * \% BG + ( - 4.466 ) * \% AF + 102.7 + ( - 2.036 ) * \% HM ,$$

wherein % BG is the % β-galactosylated glycan content, % AF is the % afucosylated glycan content, and % HM is the % high mannose glycan content.

In exemplary aspects, the FcγRIIb binding level of an antibody composition is a level within the 95% confidence interval of a line of Equation 6:

$$[ Equation 6 ]$$

$$FcyRII binding = 0.461 * \% BG + ( - 4.429 ) * \% AF + 105.731 + ( - 1.883 ) * \% HM ,$$

wherein % BG is the % β-galactosylated glycan content, % AF is the % afucosylated glycan content, and % HM is the % high mannose glycan content.

In exemplary aspects, the FcγRIIb binding level of an antibody composition is a level within the 95% confidence interval of a line of Equation 10:

$$[ Equation 10 ]$$

$$FcyRII binding = 0.59 * \% BG + ( - 2.04 ) * \% AF + 99.2 + ( - 1.91 ) * \% HM ,$$

wherein % BG is the % β-galactosylated glycan content, % AF is the % afucosylated glycan content, and % HM is the % high mannose glycan content.

In exemplary embodiments, the presently disclosed method of producing an antibody composition comprises (a) determining the afucosylated glycan content and/or β-galactosylated glycan content of a sample of the antibody composition; (b) determining the FcγRII binding level of the antibody composition based on afucosylated glycan content and/or β-galactosylated glycan content determined in (a); and (c) selecting the antibody composition for downstream processing based on the level of FcγRII binding determined in (b). In various instances, the antibody of the antibody composition comprises a chimeric constant region. In exemplary instances, the chimeric constant region of the antibody of the antibody composition comprises a portion of an IgG2 constant region and a portion of an IgG4 constant region. In various aspects, the chimeric constant region comprises CH1 and/or a hinge of an IgG2 and/or CH2-CH3 of an IgG4. In exemplary instances, the chimeric constant region comprises a chimeric constant region of SEQ ID NO: 15.

In various instances, the antibody composition comprises an anti-C5 antibody comprising the heavy chain and light chain of eculizumab. Optionally, the sample is of a cell culture comprising glycosylation-competent cells expressing an antibody of the antibody composition. In exemplary aspects, the method further comprises modifying one or more conditions of the cell culture to modify the afucosylated glycan content and/or the β-galactosylated glycan content of the antibody composition and determining the afucosylated glycan content and/or the β-galactosylated glycan content of a sample of the antibody composition taken from the modified cell culture. In exemplary instances, the method further comprises modifying one or more conditions of the cell culture to increase the level of afucosylated glycans of the antibody composition to decrease the level of FcγRII binding of the antibody composition and/or modifying one or more conditions of the cell culture to decrease the level of β-galactosylated glycans of the antibody composition to decrease the level of FcγRII binding of the antibody composition. Optionally, the method further comprises modifying one or more conditions of the cell culture to decrease the level of afucosylated glycans of the antibody composition to increase the level of FcγRII binding of the antibody composition and/or modifying one or more conditions of the cell culture to increase the level of β-galactosylated glycans of the antibody composition to increase the level of FcγRII binding of the antibody composition. In exemplary aspects, the method further comprises repeating said modifying until the afucosylated glycan content and/or the β-galactosylated glycan content is within a target range. In exemplary instances, the afucosylated glycan content and/or the β-galactosylated glycan content is/are determined in real time with respect to production of the antibody composition. In exemplary aspects, the method comprises selecting the antibody composition for downstream processing when the afucosylated glycan content and/or the β-galactosylated glycan content is/are in a target range. Optionally, the method comprises selecting the antibody composition for downstream processing when the FcγRII binding level is in a target range. In various instances, the determining the level of FcγRII binding comprises determining a level of ADCC, ADCP, and/or CDC. In various instances, the method further comprises specifying a level of ADCC, ADCP, and/or CDCC of the antibody composition, wherein the selected antibody composition comprises the specified level of ADCC, ADCP, and/or CDC

### Processing Steps

The % afucosylated glycans and/or the % β-galactosylated glycan content are determined (e.g., measured) to better inform as to the FcγRII binding level of the antibody composition. The determining (e.g., measuring) may occur at any point during manufacture. In particular, measurements may be taken pre- or post-harvest, at any stage during downstream processing, such as following any chromatography unit operation, including capture chromatography, intermediate chromatography, and/or polish chromatography unit operations; virus inactivation and neutralization, virus filtration; and/or final formulation. The % afucosylated glycans and/or the % β-galactosylated glycan content in various aspects is determined (e.g., measured) in real-time, near real-time, and/or after the fact. Monitoring and measurements can be done using known techniques and commercially available equipment.

In various aspects of the present disclosure, determining (e.g., measuring) the % afucosylated glycans and/or the % β-galactosylated glycan content is carried out after a harvest. As used herein the term “harvest” refers to the action during which cell culture media containing the recombinant protein of interest is collected and separated at least from the cells of the cell culture. Harvest can be performed continuously. The harvest in some aspects is performed using centrifugation and can further comprise precipitation, filtration, and the like. In various aspects, the determining is carried out after chromatography, optionally, Protein A chromatography. In various aspects, the determining is carried out after harvest and after chromatography, e.g., Protein A chromatography.

With regard to the presently disclosed methods, the antibody composition in various aspects is selected or chosen for further processing steps, e.g., for one or more downstream processing steps, and the selection is based on a particular parameter, e.g., % FcγRII binding, % afucosylated glycans and/or the % β-galactosylated glycan content. In various instances, the presently disclosed methods comprise using the antibody composition in further processing steps, e.g., in one or more downstream processing steps, based on a particular parameter, e.g., based on the % FcγRII binding, % afucosylated glycans, and/or the % β-galactosylated glycan content. In various instances, the presently disclosed methods comprise carrying out further processing steps, e.g., one or more downstream processing steps, with the antibody composition, based on a particular parameter, e.g., based on the % FcγRII binding, % afucosylated glycans, and/or the % β-galactosylated glycan content. Optionally, the processing steps may be performed sequentially, simultaneously, and/or may overlap with each other.

In exemplary instances the one or more downstream processing steps is any processing step which occurs after (or downstream of) the processing step at which the % afucosylated glycans and/or the % β-galactosylated glycan content is/are determined (e.g., measured). For instance, if the % afucosylated glycans and/or the % β-galactosylated glycan content were determined (e.g., measured) were determined (e.g., measured) at harvest, then the one or more downstream processing steps is any processing step which occurs after (or downstream of) the harvest step, which in various aspects comprise(s): a dilution step, a filling step, a filtration step, a formulation step, a chromatography step, a viral filtration step, a viral inactivation step, or a combination thereof. Also, for example, if the % afucosylated glycans and/or the % β-galactosylated glycan content were determined (e.g., measured) after chromatography, e.g., a Protein A chromatography, then the one or more downstream processing steps is any processing step which occurs after (or downstream of) the chromatography, which in various aspects comprise(s): a dilution step, a filling step, a filtration step, a formulation step, a further chromatography step, a viral filtration step, a viral inactivation step, or a combination thereof. In exemplary instances the further chromatography is ion exchange chromatography (e.g., a cation exchange chromatography or an anion exchange chromatography). Optionally, the downstream processing steps may be performed sequentially, simultaneously, and/or may overlap with each other.

Stages/types of chromatography used during downstream processing include capture or affinity chromatography which is used to separate the recombinant product from other proteins, aggregates, DNA, viruses and other such impurities. In exemplary instances, initial chromatography is carried out with Protein A (e.g., Protein A attached to a resin). Intermediate and polish chromatography in various aspects further purify the recombinant protein, removing bulk contaminants, adventitious viruses, trace impurities, aggregates, isoforms, etc. The chromatography can either be performed in bind and elute mode, where the recombinant protein of interest is bound to the chromatography medium and the impurities flow through, or in flow-through mode, where the impurities are bound and the recombinant protein flows through. Examples of such chromatography methods include ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography and gel filtration.

In various aspects, the downstream step is a viral inactivation step. Enveloped viruses have a capsid enclosed by a lipoprotein membrane or “envelope” and are therefore susceptible to inactivation. The virus inactivation step in various instances includes heat inactivation/pasteurization, pH inactivation, UV and gamma ray irradiation, use of high intensity broad spectrum white light, addition of chemical inactivating agents, surfactants, and solvent/detergent treatments.

In various aspects, the downstream step is a virus filtration step. In various aspects, the virus filtration step comprises removing non-enveloped viruses. In various aspects, the virus filtration step comprises the use of micro- or nano-filters.

In various aspects, the downstream processing step comprises one or more formulation steps. Following completion of the chromatography steps, the purified recombinant proteins are in various aspects buffer exchanged into a formulation buffer. In exemplary aspects, the buffer exchange is performed using ultrafiltration and diafiltration (UF/DF). In exemplary aspects, the recombinant protein is buffer exchanged into a desired formulation buffer using diafiltration and concentrated to a desired final formulation concentration using ultrafiltration. Additional stability-enhancing excipients in various aspects are added following a UF/DF formulation step.

### Recombinant Glycosylated Proteins

The presently disclosed methods relate to composition comprising a recombinant glycosylated protein. In various aspects, the recombinant glycosylated protein comprises an amino acid sequence comprising one or more N-glycosylation consensus sequences of the formula:

Asn-Xaa₁-Xaa₂

wherein Xaa₁ is any amino acid except Pro, and Xaa₂ is Ser or Thr.

In exemplary embodiments, the recombinant glycosylated protein comprises a fragment crystallizable (Fc) polypeptide. The term “Fc polypeptide” as used herein includes native and mutein forms of polypeptides derived from the Fc region of an antibody. Truncated forms of such polypeptides containing the hinge region that promotes dimerization also are included. Fusion proteins comprising Fc moieties (and oligomers formed therefrom) offer the advantage of facile purification by affinity chromatography over Protein A or Protein G columns. In exemplary embodiments, the recombinant glycosylated protein comprises the Fc of an IgG, e.g., a human IgG. In exemplary aspects, the recombinant glycosylated protein comprises the Fc an IgG1 or IgG2. In exemplary aspects, the recombinant glycosylated protein is an antibody, an antibody protein product, a peptibody, or a Fc-fusion protein.

In exemplary aspects, the recombinant glycosylated protein is an antibody. As used herein, the term “antibody” refers to a protein having a conventional immunoglobulin format, comprising heavy and light chains, and comprising variable and constant regions. For example, an antibody may be an IgG which is a ‘Y-shaped’ structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. See, e.g., Janeway et al., “Structure of the Antibody Molecule and the Immunoglobulin Genes %, Immunobiology: The Immune System in Health and Disease, 4^{th} ed. Elsevier Science Ltd/Garland Publishing, (1999).

Briefly, in an antibody scaffold, the CDRs are embedded within a framework in the heavy and light chain variable region where they constitute the regions largely responsible for antigen binding and recognition. A variable region comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

In exemplary aspects, the recombinant glycosylated protein (such as an antibody) comprises a chimeric constant region. In exemplary instances, the chimeric constant region of the recombinant glycosylated protein comprises a portion of an IgG2 constant region and a portion of an IgG4 constant region. In various aspects, the chimeric constant region comprises CH1 and/or a hinge of an IgG2 and/or CH2-CH3 of an IgG4. In exemplary instances, the chimeric constant region comprises a chimeric constant region of SEQ ID NO: 15. The recombinant glycosylated protein may be the antibody of an antibody composition as described herein.

In various aspects, the antibody can be a monoclonal antibody or a polyclonal antibody. In exemplary instances, the antibody is a mammalian antibody, e.g., a mouse antibody, rat antibody, rabbit antibody, goat antibody, horse antibody, chicken antibody, hamster antibody, pig antibody, human antibody, and the like. In certain aspects, the recombinant glycosylated protein is a monoclonal human antibody.

An antibody, in various aspects, is cleaved into fragments by enzymes, such as, e.g., papain and pepsin. Papain cleaves an antibody to produce two Fab fragments and a single Fc fragment. Pepsin cleaves an antibody to produce a F(ab′)₂ fragment and a pFc′ fragment. In exemplary aspects, the recombinant glycosylated protein is an antibody fragment, e.g., a Fab, Fc, F(ab′)₂, or a pFc′, that retains at least one glycosylation site. With regard to the methods of the disclosure, the antibody may lack certain portions of an antibody, and may be an antibody fragment. In various aspects, the antibody fragment comprises a glycosylation site. In some aspects, the fragment is a “Glycosylated Fc Fragment” which comprises at least a portion of the Fc region of an antibody which is glycosylated post-translationally in eukaryotic cells. In various instances, the recombinant glycosylated protein is glycosylated Fc fragment.

The architecture of antibodies has been exploited to create a growing range of alternative antibody formats that spans a molecular-weight range of at least or about 12-150 kDa and a valency (n) range from monomeric (n=1), dimeric (n=2) and trimeric (n=3) to tetrameric (n=4) and potentially higher, such alternative antibody formats are referred to herein as “antibody protein products” or “antibody binding proteins”.

Antibody protein products can be an antigen binding format based on antibody fragments, e.g., scFvs, Fabs and VHHNH, which retain full antigen-binding capacity. The smallest antigen-binding fragment that retains its complete antigen binding site is the Fv fragment, which consists entirely of variable (V) regions. A soluble, flexible amino acid peptide linker is used to connect the V regions to a scFv (single chain fragment variable) fragment for stabilization of the molecule, or the constant (C) domains are added to the V regions to generate a Fab fragment [fragment, antigen-binding]. Both scFv and Fab are widely used fragments that can be easily produced in prokaryotic hosts. Other antibody protein products include disulfide-bond stabilized scFv (ds-scFv), single chain Fab (scFab), as well as di- and multimeric antibody formats like dia-, tria- and tetra-bodies, or minibodies (miniAbs) that comprise different formats consisting of scFvs linked to oligomerization domains. The smallest fragments are VHHNH of camelid heavy chain Abs as well as single domain Abs (sdAb). The building block that is most frequently used to create novel antibody formats is the single-chain variable (V)-domain antibody fragment (scFv), which comprises V domains from the heavy and light chain (VH and VL domain) linked by a peptide linker of ˜15 amino acid residues. A peptibody or peptide-Fc fusion is yet another antibody protein product. The structure of a peptibody consists of a biologically active peptide grafted onto an Fc domain. Peptibodies are well-described in the art. See, e.g., Shimamoto et al., mAbs 4(5): 586-591 (2012).

Other antibody protein products include a single chain antibody (SCA); a diabody; a triabody; a tetrabody; bispecific or trispecific antibodies, and the like. Bispecific antibodies can be divided into five major classes: BsIgG, appended IgG, BsAb fragments, bispecific fusion proteins and BsAb conjugates. See, e.g., Spiess et al., Molecular Immunology 67(2) Part A: 97-106 (2015).

In exemplary aspects, the recombinant glycosylated protein comprises any one of these antibody protein products (e.g., scFv, Fab VHHNH, Fv fragment, ds-scFv, scFab, dimeric antibody, multimeric antibody (e.g., a diabody, triabody, tetrabody), miniAb, peptibody VHHNH of camelid heavy chain antibody, sdAb, diabody; a triabody; a tetrabody; a bispecific or trispecific antibody, BsIgG, appended IgG, BsAb fragment, bispecific fusion protein, and BsAb conjugate) and comprises one or more N-glycosylation consensus sequences, optionally, one or more Fc polypeptides. In various aspects, the antibody protein product comprises a glycosylation site. In exemplary aspects, an antibody protein product can be a Glycosylated Fc Fragment conjugated to an antibody binding fragment (“Glycosylated Fc Fragment antibody product”).

The recombinant glycosylated protein may be an antibody protein product in monomeric form, or polymeric, oligomeric, or multimeric form. In certain embodiments in which the antibody comprises two or more distinct antigen binding regions fragments, the antibody is considered bispecific, trispecific, or multi-specific, or bivalent, trivalent, or multivalent, depending on the number of distinct epitopes that are recognized and bound by the antibody.

In various aspects, the recombinant glycosylated protein is a chimeric antibody or a humanized antibody. The term “chimeric antibody” is used herein to refer to an antibody containing constant domains from one species and the variable domains from a second, or more generally, containing stretches of amino acid sequence from at least two species. The term “humanized” when used in relation to antibodies refers to antibodies having at least CDR regions from a non-human source which are engineered to have a structure and immunological function more similar to true human antibodies than the original source antibodies. For example, humanizing can involve grafting CDR from a non-human antibody, such as a mouse antibody, into a human antibody. Humanizing also can involve select amino acid substitutions to make a non-human sequence look more like a human sequence.

Advantageously, the methods are not limited to an antigen-specificity of the antibody, glycosylated Fc fragment, antibody protein product, chimeric antibody, or humanized antibody. Accordingly, the antibody, glycosylated Fc fragment, antibody protein product, chimeric antibody, or humanized antibody has any binding specificity for virtually any antigen. In exemplary aspects, the antibody binds to a hormone, growth factor, cytokine, a cell-surface receptor, or any ligand thereof. In exemplary aspects, the antibody binds to a protein expressed on the cell surface of an immune cell. In exemplary aspects, the antibody binds to a cluster of differentiation molecule selected from the group consisting of: CD1a, CD1b, CD1c, CD1d, CD2, CD3, CD4, CD5, CD6, CD7, CD8, CD9, CD10, CD11A, CD11B, CD11C, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L, CD62P, CD63, CD64, CD65, CD66a, CD66b, CD66c, CD66d, CD66e, CD66f, CD68, CD69, CD70, CD71, CD72, CD73, CD74, CD75, CD76, CD79a, CD79p, CD80, CD81, CD82, CD83, CDw84, CD85, CD86, CD87, CD88, CD89, CD90, CD91, CDw92, CD93, CD94, CD95, CD96, CD97, CD98, CD99, CD100, CD101, CD102, CD103, CD104, CD105, CD106, CD107a, CD107b, CDw108, CD109, CD114, CD115, CD116, CD117, CD118, CD119, CD120a, CD120b, CD121a, CDw121b, CD122, CD123, CD124, CD125, CD126, CD127, CDw128, CD129, CD130, CDw131, CD132, CD134, CD135, CDw136, CDw137, CD138, CD139, CD140a, CD140b, CD141, CD142, CD143, CD144, CD145, CD146, CD147, CD148, CD150, CD151, CD152, CD153, CD154, CD155, CD156, CD157, CD158a, CD158b, CD161, CD162, CD163, CD164, CD165, CD166, and CD182.

In exemplary aspects, the antibody, glycosylated Fc fragment, antibody protein product, chimeric antibody, or humanized antibody is one of those described in U.S. Pat. No. 7,947,809 and U.S. Patent Application Publication No. 20090041784 (glucagon receptor), U.S. Pat. Nos. 7,939,070, 7,833,527, 7,767,206, and 7,786,284 (IL-17 receptor A), U.S. Pat. Nos. 7,872,106 and 7,592,429 (Sclerostin), U.S. Pat. Nos. 7,871,611, 7,815,907, 7,037,498, 7,700,742, and U.S. Patent Application Publication No. 20100255538 (IGF-1 receptor), U.S. Pat. No. 7,868,140 (B7RP1), U.S. Pat. No. 7,807,159 and U.S. Patent Application Publication No. 20110091455 (myostatin), U.S. Pat. Nos. 7,736,644, 7,628,986, 7,524,496, and U.S. Patent Application Publication No. 20100111979 (deletion mutants of epidermal growth factor receptor), U.S. Pat. No. 7,728,110 (SARS coronavirus), U.S. Pat. No. 7,718,776 and U.S. Patent Application Publication No. 20100209435 (OPGL), U.S. Pat. Nos. 7,658,924 and 7,521,053 (Angiopoietin-2), U.S. Pat. Nos. 7,601,818, 7,795,413, U.S. Patent Application Publication No. 20090155274, U.S. Patent Application Publication No. 20110040076 (NGF), U.S. Pat. No. 7,579,186 (TGF-β type II receptor), U.S. Pat. No. 7,541,438 (connective tissue growth factor), U.S. Pat. No. 7,438,910 (IL1-R1), U.S. Pat. No. 7,423,128 (properdin), U.S. Pat. Nos. 7,411,057, 7,824,679, 7,109,003, 6,682,736, 7,132,281, and 7,807,797 (CTLA-4), U.S. Pat. Nos. 7,084,257, 7,790,859, 7,335,743, 7,084,257, and U.S. Patent Application Publication No. 20110045537 (interferon-gamma), U.S. Pat. No. 7,932,372 (MAdCAM), U.S. Pat. No. 7,906,625, U.S. Patent Application Publication No. 20080292639, and U.S. Patent Application Publication No. 20110044986 (amyloid), U.S. Pat. Nos. 7,815,907 and 7,700,742 (insulin-like growth factor 1), U.S. Pat. Nos. 7,566,772 and 7,964,193 (interleukin-10), U.S. Pat. Nos. 7,563,442, 7,288,251, 7,338,660, 7,626,012, 7,618,633, and U.S. Patent Application Publication No. 20100098694 (CD40), U.S. Pat. No. 7,498,420 (c-Met), U.S. Pat. Nos. 7,326,414, 7,592,430, and 7,728,113 (M-CSF), U.S. Pat. Nos. 6,924,360, 7,067,131, and 7,090,844 (MUC18), U.S. Pat. Nos. 6,235,883, 7,807,798, and U.S. Patent Application Publication No. 20100305307 (epidermal growth factor receptor), U.S. Pat. Nos. 6,716,587, 7,872,113, 7,465,450, 7,186,809, 7,317,090, and 7,638,606 (interleukin-4 receptor), U.S. Patent Application Publication No. 20110135657 (BETA-KLOTHO), U.S. Pat. Nos. 7,887,799 and 7,879,323 (fibroblast growth factor-like polypeptides), U.S. Pat. No. 7,867,494 (IgE), U.S. Patent Application Publication No. 20100254975 (ALPHA-4 BETA-7), U.S. Patent Application Publication No. 20100197005 and U.S. Pat. No. 7,537,762 (ACTIVIN RECEPTOR-LIKE KINASE-1), U.S. Pat. No. 7,585,500 and U.S. Patent Application Publication No. 20100047253 (IL-13), U.S. Patent Application Publication No. 20090263383 and U.S. Pat. No. 7,449,555 (CD148), U.S. Patent Application Publication No. 20090234106 (ACTIVIN A), U.S. Patent Application Publication No. 20090226447 (angiopoietin-1 and angiopoietin-2), U.S. Patent Application Publication No. 20090191212 (Angiopoietin-2), U.S. Patent Application Publicaiton No. 20090155164 (C-FMS), U.S. Pat. No. 7,537,762 (activin receptor-like kinase-1), U.S. Pat. No. 7,371,381 (galanin), U.S. Patent Application Publication No. 20070196376 (INSULIN-LIKE GROWTH FACTORS), U.S. Pat. Nos. 7,267,960 and 7,741,115 (LDCAM), U.S. Pat. No. 7,265,212 (CD45RB), U.S. Pat. No. 7,709,611, U.S. Patent Application Publication No. 20060127393 and U.S. Patent Application Publication No. 20100040619 (DKK1), U.S. Pat. No. 7,807,795, U.S. Patent Application Publication No. 20030103978 and U.S. Pat. No. 7,923,008 (osteoprotegerin), U.S. Patent Application Publication No. 20090208489 (OV064), U.S. Patent Application Publication No. 20080286284 (PSMA), U.S. Pat. No. 7,888,482, U.S. Patent Application Publication No. 20110165171, and U.S. Patent Application Publication No. 20110059063 (PAR2), U.S. Patent Application Publication No. 20110150888 (HEPCIDIN), U.S. Pat. No. 7,939,640 (B7L-1), U.S. Pat. No. 7,915,391 (c-Kit), U.S. Pat. Nos. 7,807,796, 7,193,058, and U.S. Pat. No. 7,427,669 (ULBP), U.S. Pat. Nos. 7,786,271, 7,304,144, and U.S. Patent Application Publication No. 20090238823 (TSLP), U.S. Pat. No. 7,767,793 (SIGIRR), U.S. Pat. No. 7,705,130 (HER-3), U.S. Pat. No. 7,704,501 (ataxin-1-like polypeptide), U.S. Pat. Nos. 7,695,948 and 7,199,224 (TNF-α converting enzyme), U.S. Patent Application Publication No. 20090234106 (ACTIVIN A), U.S. Patent Application Publication No. 20090214559 and U.S. Pat. No. 7,438,910 (IL1-R1), U.S. Pat. No. 7,579,186 (TGF-β type II receptor), U.S. Pat. No. 7,569,387 (TNF receptor-like molecules), U.S. Pat. No. 7,541,438, (connective tissue growth factor), U.S. Pat. No. 7,521,048 (TRAIL receptor-2), U.S. Pat. Nos. 6,319,499, 7,081,523, and U.S. Patent Application Publication No. 20080182976 (erythropoietin receptor), U.S. Patent Application Publication No. 20080166352 and U.S. Pat. No. 7,435,796 (B7RP1), U.S. Pat. No. 7,423,128 (properdin), U.S. Pat. Nos. 7,422,742 and 7,141,653 (interleukin-5), U.S. Pat. Nos. 6,740,522 and 7,411,050 (RANKL), U.S. Pat. No. 7,378,091 (carbonic anhydrase IX (CA IX) tumor antigen), U.S. Pat. Nos. 7,318,925 and 7,288,253 (parathyroid hormone), U.S. Pat. No. 7,285,269 (TNF), U.S. Pat. Nos. 6,692,740 and 7,270,817 (ACPL), U.S. Pat. No. 7,202,343 (monocyte chemo-attractant protein-1), U.S. Pat. No. 7,144,731 (SCF), U.S. Pat. Nos. 6,355,779 and 7,138,500 (4-1BB), U.S. Pat. No. 7,135,174 (PDGFD), U.S. Pat. Nos. 6,630,143 and 7,045,128 (Flt-3 ligand), U.S. Pat. No. 6,849,450 (metalloproteinase inhibitor), U.S. Pat. No. 6,596,852 (LERK-5), U.S. Pat. No. 6,232,447 (LERK-6), U.S. Pat. No. 6,500,429 (brain-derived neurotrophic factor), U.S. Pat. No. 6,184,359 (epithelium-derived T-cell factor), U.S. Pat. No. 6,143,874 (neurotrophic factor NNT-1), U.S. Patent Application Publication No. 20110027287 (PROPROTEIN CONVERTASE SUBTILISIN KEXIN TYPE 9 (PCSK9)), U.S. Patent Application Publication No. 20110014201 (IL-18 RECEPTOR), and U.S. Patent Application Publication No. 20090155164 (C-FMS). The above patents and published patent applications are incorporated herein by reference in their entirety for purposes of their disclosure of variable domain polypeptides, variable domain encoding nucleic acids, host cells, vectors, methods of making polypeptides encoding said variable domains, pharmaceutical compositions, and methods of treating diseases associated with the respective target of the variable domain-containing antigen binding protein or antibody.

In exemplary embodiments, the antibody, glycosylated Fc fragment, antibody protein product, chimeric antibody, or humanized antibody is one of Muromonab-CD3 (product marketed with the brand name Orthoclone Okt3®), Abciximab (product marketed with the brand name Reopro®.), Rituximab (product marketed with the brand name MabThera®, Rituxan®), Basiliximab (product marketed with the brand name Simulect®), Daclizumab (product marketed with the brand name Zenapax®), Palivizumab (product marketed with the brand name Synagis®), Infliximab (product marketed with the brand name Remicade®), Trastuzumab (product marketed with the brand name Herceptin®), Alemtuzumab (product marketed with the brand name MabCampath®, Campath-1H®), Adalimumab (product marketed with the brand name Humira®), Tositumomab-1131 (product marketed with the brand name Bexxar®), Efalizumab (product marketed with the brand name Raptiva®), Cetuximab (product marketed with the brand name Erbitux®), l'Ibritumomab tiuxetan (product marketed with the brand name Zevalin®), l'Omalizumab (product marketed with the brand name Xolair®), Bevacizumab (product marketed with the brand name Avastin®), Natalizumab (product marketed with the brand name Tysabri®), Ranibizumab (product marketed with the brand name Lucentis®), Panitumumab (product marketed with the brand name Vectibix®), Eculizumab (product marketed with the brand name Soliris®), Certolizumab pegol (product marketed with the brand name Cimzia®), Golimumab (product marketed with the brand name Simponi®), Canakinumab (product marketed with the brand name Ilaris®), Catumaxomab (product marketed with the brand name Removab®), Ustekinumab (product marketed with the brand name Stelara®), Tocilizumab (product marketed with the brand name RoActemra®, Actemra®), Ofatumumab (product marketed with the brand name Arzerra®), Denosumab (product marketed with the brand name Prolia®), Belimumab (product marketed with the brand name Benlysta®), Raxibacumab, Ipilimumab (product marketed with the brand name Yervoy®), and Pertuzumab (product marketed with the brand name Perjeta®). In exemplary embodiments, the antibody is one of anti-TNF alpha antibodies such as adalimumab, infliximab, etanercept, golimumab, and certolizumab pegol; anti-IL1.beta. antibodies such as canakinumab; anti-IL12/23 (p40) antibodies such as ustekinumab and briakinumab; and anti-IL2R antibodies, such as daclizumab.

In exemplary aspects, the antigen of the antibody is Complement protein C5, e.g., human complement C5, and the antibody is an anti-C5 antibody, e.g., an anti-human C5 monoclonal antibody. C5 is a component of the complement system which is a part of the innate immune system. The C5 preproprotein is proteolytically processed to produce multiple protein products, including the C5 alpha chain, C5 beta chain, C5a anaphylatoxin and C5b. The C5 protein is comprised of the C5 alpha and beta chains, which are linked by a disulfide bridge. The amino acid sequence of the preproprotein is provided herein as SEQ ID NO: 2 wherein residues 19-673 represent the sequence of the Complement C5 beta chain, residues 752-1676 represent the sequence of the Complement C5 alpha chain, and residues 678-751 represent the sequence of the C5a anaphylatoxin. SEQ ID NO: 3 is the sequence of the mRNA sequence of the transcript variant 1 encoded by the human C5 gene. In various aspects, the antibody is eculizumab or a biosimilar thereof. The term eculizumab refers to a chimeric monoclonal antibody comprising the hinge and CH1 domains of an IgG2 and the CH2 and CH3 domains of an IgG4, which mAb binds Complement protein C5 (See CAS Number: 219685-50, DrugBank Accession No. DB01257). In exemplary aspects, the antibody comprises a light chain comprising a CDR1, CDR2, and CDR3 of the variable region of the eculizumab light chain as set forth in Table A. In exemplary aspects, the antibody comprises a heavy chain comprising a CDR1, CDR2, and CDR3 of the variable region of the eculizumab heavy chain as set forth in Table A. In various instances, the antibody comprises the VH and VL or comprising VH-IgG1 and VL-IgG kappa sequences of eculizumab.

TABLE A
Eculizumab Amino Acid Sequences

| | | SEQ |
|---|---|---|
| | | ID |
| Description | Sequence | NO: |
| LC CDR1 | GASENIYGALN | 4 |
| LC CDR2 | GATNLAD | 5 |
| LC CDR3 | QNVLNTPLT | 6 |
| HC CDR1 | GYIFSNYWIQ | 7 |
| HC CDR2 | EILPGSGSTEYTENFKD | 8 |
| HC CDR3 | YFFGSSPNWYFDV | 9 |
| LC | DIQMTQSPSSLSASVGDRVTITOGASENIYGALNWYQQKPGKAPKLLIYGA | 10 |
| variable | TNLADGVPSRFSGSGSGTDFTLTISSLQPEDFATYYCQNVLNTPLTFGQGT | |
| region | KVEIK | |
| HC | QVQLVQSGAEVKKPGASVKVSCKASGYIFSNYWIQWVRQAPGQGLEWM | 11 |
| variable | GEILPGSGSTEYTENFKDRVTMTRDTSTSTVYMELSSLRSEDTAVYYCARY | |
| region | FFGSSPNWYFDVWGQGTLVTVSS | |
| LC | RTVAAPSVFIFPPSDEQLKSGTASVVCLLNNFYPREAKVQWKVDNALQSG | 14 |
| constant | NSQESVTEQDSKDSTYSLSSTLTLSKADYEKHKVYACEVTHQGLSSPVTKS | |
| region | FNRGEC | |
| HC | ASTKGPSVFPLAPCSRSTSESTAALGCLVKDYFPEPVTVSWNSGALTSGV | 15 |
| constant | HTFPAVLQSSGLYSLSSVVTVPSSNFGTQTYTCNVDHKPSNTKVDKTVER | |
| region | KCCVECPPCPAPPVAGPSVFLFPPKPKDTLMISRTPEVTCVVVDVSQEDPE | |
| | VQFNWYVDGVEVHNAKTKPREEQFNSTYRVVSVLTVLHQDWLNGKEYKC | |
| | KVSNKGLPSSIEKTISKAKGQPREPQVYTLPPSQEEMTKNQVSLTCLVKGF | |
| | YPSDIAVEWESNGQPENNYKTTPPVLDSDGSFFLYSRLTVDKSRWQEGNV | |
| | FSCSVMHEALHNHYTQKSLSLSLGK | |
| FL Light | DIQMTQSPSSLSASVGDRVTITCGASENIYGALNWYQQKPGKAPKLLIYGA | 12 |
| Chain | TNLADGVPSRFSGSGSGTDFTLTISSLQPEDFATYYCQNVLNTPLTFGQGT | |
| | KVEIKRTVAAPSVFIFPPSDEQLKSGTASVVCLLNNFYPREAKVQWKVDNA | |
| | LQSGNSQESVTEQDSKDSTYSLSSTLTLSKADYEKHKVYACEVTHQGLSS | |
| | PVTKSFNRGEC | |
| FL Heavy | QVQLVQSGAEVKKPGASVKVSCKASGYIFSNYWIQWVRQAPGQGLEWM | 13 |
| Chain | GEILPGSGSTEYTENFKDRVTMTROTSTSTVYMELSSLRSEDTAVYYCARY | |
| | FFGSSPNWYFDVWGQGTLVTVSSASTKGPSVFPLAPCSRSTSESTAALGC | |
| | LVKDYFPEPVTVSWNSGALTSGVHTFPAVLQSSGLYSLSSVVTVPSSNFGT | |
| | QTYTCNVDHKPSNTKVDKTVERKCCVECPPCPAPPVAGPSVFLFPPKPKD | |
| | TLMISRTPEVTCVVVDVSQEDPEVQFNWYVDGVEVHNAKTKPREEQFNST | |
| | YRVVSVLTVLHQDWLNGKEYKCKVSNKGLPSSIEKTISKAKGQPREPQVYT | |
| | LPPSQEEMTKNQVSLTCLVKGFYPSDIAVEWESNGQPENNYKTTPPVLDS | |
| | DGSFFLYSRLTVDKSRWQEGNVFSCSVMHEALHNHYTQKSLSLSLGK | |

LC, light chain; HC, heavy chain; CDR, complementarity determining region.
Bold and underlined sequence of SEQ ID NO: 15 identifies a hinge of an IgG2 and the sequence N-terminal to the hinge is a CH1 of an IgG2 (Hougs et al., Immunogenetics 52: 242-248 (2001)); italicized sequence identifies CH2-CH3 of an IgG4 (Uniprot P01861).

In various aspects, the antibody comprises:
i. a light chain (LC) CDR1 comprising an amino acid sequence of SEQ ID NO: 4 or an amino acid sequence which is at least 90% (e.g., at least 95%, at least 96%, at least 97%, at least 98% or at least 99%) identical to SEQ ID NO: 4 or a variant amino acid sequence of SEQ ID NO: 4 with 1 or 2 amino acid substitutions,
ii. a LC CDR2 comprising an amino acid sequence of SEQ ID NO: 5 or an amino acid sequence which is at least 90% (e.g., at least 95%, at least 96%, at least 97%, at least 98% or at least 99%) identical to SEQ ID NO: 5 or a variant amino acid sequence of SEQ ID NO: 5 with 1 or 2 amino acid substitutions,
iii. a LC CDR3 comprising an amino acid sequence of SEQ ID NO: 6 or an amino acid sequence which is at least 90% (e.g., at least 95%, at least 96%, at least 97%, at least 98% or at least 99%) identical to SEQ ID NO: 6 or a variant amino acid sequence of SEQ ID NO: 6 with 1 or 2 amino acid substitutions,
iv. a heavy chain (HC) CDR1 comprising an amino acid sequence of SEQ ID NO: 7 or an amino acid sequence which is at least 90% (e.g., at least 95%, at least 96%, at least 97%, at least 98% or at least 99%) identical to SEQ ID NO: 7 or a variant amino acid sequence of SEQ ID NO: 7 with 1 or 2 amino acid substitutions;
v. a HC CDR2 comprising an amino acid sequence of SEQ ID NO: 8 or an amino acid sequence which is at least 90% (e.g., at least 95%, at least 96%, at least 97%, at least 98% or at least 99%) identical to SEQ ID NO: 8 or a variant amino acid sequence of SEQ ID NO: 8 with 1 or 2 amino acid substitutions;
vi. a HC CDR3 comprising an amino acid sequence of SEQ ID NO: 9 or an amino acid sequence which is at least 90% (e.g., at least 95%, at least 96%, at least 97%, at least 98% or at least 99%) identical to SEQ ID NO: 9 or a variant amino acid sequence of SEQ ID NO: 9 with 1 or 2 amino acid substitutions.

In various instances, the antibody comprises: a LC variable region comprising an amino acid sequence of SEQ ID NO: 10, an amino acid sequence which is at least 90% (e.g., at least 95%, at least 96%, at least 97%, at least 98% or at least 99%) identical to SEQ ID NO: 10, or a variant amino acid sequence of SEQ ID NO: 10 with 1 to 10 (e.g., 1 to 9, 1 to 8, 1 to 7, 1 to 6, 1 to 5, 1 to 4, 1 to 3, 1 or 2) amino acid substitutions.

In exemplary aspects, the antibody comprises: a HC variable region comprising an amino acid sequence of SEQ ID NO: 11, an amino acid sequence which is at least 90% (e.g., at least 95%, at least 96%, at least 97%, at least 98% or at least 99%) identical to SEQ ID NO: 11, or a variant amino acid sequence of SEQ ID NO: 11 with 1 to 10 (e.g., 1 to 9, 1 to 8, 1 to 7, 1 to 6, 1 to 5, 1 to 4, 1 to 3, 1 or 2) amino acid substitutions.

In exemplary instances, the antibody comprises a light chain comprising an amino acid sequence of SEQ ID NO: 12, an amino acid sequence which is at least 90% (e.g., at least 95%, at least 96%, at least 97%, at least 98% or at least 99%) identical to SEQ ID NO: 12, or a variant amino acid sequence of SEQ ID NO: 12 with 1 to 10 (e.g., 1 to 9, 1 to 8, 1 to 7, 1 to 6, 1 to 5, 1 to 4, 1 to 3, 1 or 2) amino acid substitutions.

In various aspects, the antibody comprises a heavy chain comprising an amino acid sequence of SEQ ID NO: 13, an amino acid sequence which is at least 90% (e.g., at least 95%, at least 96%, at least 97%, at least 98% or at least 99%) identical to SEQ ID NO: 13, or a variant amino acid sequence of SEQ ID NO: 13 with 1 to 10 (e.g., 1 to 9, 1 to 8, 1 to 7, 1 to 6, 1 to 5, 1 to 4, 1 to 3, 1 or 2) amino acid substitutions.

In exemplary instances, the antibody comprises a light chain constant region comprising an amino acid sequence of SEQ ID NO: 14, an amino acid sequence which is at least 90% (e.g., at least 95%, at least 96%, at least 97%, at least 98% or at least 99%) identical to SEQ ID NO: 14, or a variant amino acid sequence of SEQ ID NO: 14 with 1 to 10 (e.g., 1 to 9, 1 to 8, 1 to 7, 1 to 6, 1 to 5, 1 to 4, 1 to 3, 1 or 2) amino acid substitutions.

In various aspects, the antibody comprises a heavy chain constant region comprising an amino acid sequence of SEQ ID NO: 15, an amino acid sequence which is at least 90% (e.g., at least 95%, at least 96%, at least 97%, at least 98% or at least 99%) identical to SEQ ID NO: 15, or a variant amino acid sequence of SEQ ID NO: 15 with 1 to 10 (e.g., 1 to 9, 1 to 8, 1 to 7, 1 to 6, 1 to 5, 1 to 4, 1 to 3, 1 or 2) amino acid substitutions.

### Compositions

The presently disclosed methods relate to compositions comprising recombinant glycosylated proteins. In various aspects, the composition comprises only one type of recombinant glycosylated protein. In various instances, the composition comprises recombinant glycosylated proteins wherein each recombinant glycosylated protein of the composition comprises the same or essentially the amino acid sequence. In various aspects, the composition comprises recombinant glycosylated proteins wherein each recombinant glycosylated protein of the composition comprises an amino acid sequence which is at least 90% identical to the amino acid sequences of all other recombinant glycosylated proteins of the composition. In various aspects, the composition comprises recombinant glycosylated proteins wherein each recombinant glycosylated protein of the composition comprises an amino acid sequence which is at least 95%, at least 96%, at least 97%, at least 98%, or at least 99% identical to the amino acid sequences of all other recombinant glycosylated proteins of the composition. In various aspects, the composition comprises recombinant glycosylated proteins wherein each recombinant glycosylated protein of the composition comprises an amino acid sequence which is the same or essentially the same (e.g., at least 90% or at least 95%, at least 96%, at least 97%, at least 98%, or at least 99% identical to the amino acid sequences of all other recombinant glycosylated proteins of the composition) but the glycoprofiles of the recombinant glycosylated proteins of the composition may differ from each other.

In exemplary aspects, the recombinant glycosylated protein is an antibody fragment and accordingly, the composition may be an antibody fragment composition.

In exemplary aspects, the recombinant glycosylated protein is an antibody protein product and accordingly, the composition may be an antibody protein product composition.

In exemplary aspects, the recombinant glycosylated protein is a Glycosylated Fc Fragment and accordingly, the composition may be a Glycosylated Fc Fragment composition.

In exemplary aspects, the recombinant glycosylated protein is a Glycosylated Fc Fragment antibody product and accordingly, the composition may be a Glycosylated Fc Fragment antibody product composition.

In exemplary aspects, the recombinant glycosylated protein is a chimeric antibody and accordingly, the composition may be a chimeric antibody composition.

In exemplary aspects, the recombinant glycosylated protein is a humanized antibody and accordingly, the composition may be a humanized antibody composition.

In exemplary aspects, the recombinant glycosylated protein is an antibody and the composition is an antibody composition. In various aspects, the composition comprises only one type of antibody. In various instances, the composition comprises antibodies wherein each antibody of the antibody composition comprises the same or essentially the amino acid sequence. In various aspects, the antibody composition comprises antibodies wherein each antibody of the antibody composition comprises an amino acid sequence which is at least 90% identical to the amino acid sequences of all other antibodies of the antibody composition. In various aspects, the antibody composition comprises antibodies wherein each antibody of the antibody composition comprises an amino acid sequence which is at least 95%, at least 96%, at least 97%, at least 98%, or at least 99% identical to the amino acid sequences of all other antibodies of the antibody composition. In various aspects, the antibody composition comprises antibodies wherein each antibody of the antibody composition comprises an amino acid sequence which is the same or essentially the same (e.g., at least 90% or at least 95%, at least 96%, at least 97%, at least 98%, or at least 99% identical to the amino acid sequences of all other antibodies of the antibody composition) but the glycoprofiles of the antibodies of the antibody composition may differ from each other. In exemplary aspects, the antibody composition comprises a heterogeneous mixture of different glycoforms of the antibody. In various instances, the antibody composition may be characterized in terms of its AF glycan content and/or its β-galactosylated glycan content. In various aspects, the antibody composition is described in terms of a % AF glycan content and/or its % β-galactosylated glycan content. Optionally, the antibody composition may be characterized in terms its content of other types of glycans, e.g., high mannose glycoforms, fucosylated glycoforms, and the like.

In various aspects, each antibody of the antibody composition in an IgG, optionally, an IgG comprising a hinge and CH1 domain of an IgG2 and CH2 and CH3 domains of an IgG4. In various instances, each antibody of the antibody composition binds to complement protein C5. In exemplary aspects, each antibody of the antibody composition is an anti-C5 antibody. In various aspects, each antibody of the antibody composition comprises:
i. a light chain (LC) CDR1 comprising an amino acid sequence of SEQ ID NO: 4 or an amino acid sequence which is at least 90% (e.g., at least 95%, at least 96%, at least 97%, at least 98% or at least 99%) identical to SEQ ID NO: 4 or a variant amino acid sequence of SEQ ID NO: 4 with 1 or 2 amino acid substitutions,
ii. a LC CDR2 comprising an amino acid sequence of SEQ ID NO: 5 or an amino acid sequence which is at least 90% (e.g., at least 95%, at least 96%, at least 97%, at least 98% or at least 99%) identical to SEQ ID NO: 5 or a variant amino acid sequence of SEQ ID NO: 5 with 1 or 2 amino acid substitutions,
iii. a LC CDR3 comprising an amino acid sequence of SEQ ID NO: 6 or an amino acid sequence which is at least 90% (e.g., at least 95%, at least 96%, at least 97%, at least 98% or at least 99%) identical to SEQ ID NO: 6 or a variant amino acid sequence of SEQ ID NO: 6 with 1 or 2 amino acid substitutions,
iv. a heavy chain (HC) CDR1 comprising an amino acid sequence of SEQ ID NO: 7 or an amino acid sequence which is at least 90% (e.g., at least 95%, at least 96%, at least 97%, at least 98% or at least 99%) identical to SEQ ID NO: 7 or a variant amino acid sequence of SEQ ID NO: 7 with 1 or 2 amino acid substitutions;
v. a HC CDR2 comprising an amino acid sequence of SEQ ID NO: 8 or an amino acid sequence which is at least 90% (e.g., at least 95%, at least 96%, at least 97%, at least 98% or at least 99%) identical to SEQ ID NO: 8 or a variant amino acid sequence of SEQ ID NO: 8 with 1 or 2 amino acid substitutions; and/or
vi. a HC CDR3 comprising an amino acid sequence of SEQ ID NO: 9 or an amino acid sequence which is at least 90% (e.g., at least 95%, at least 96%, at least 97%, at least 98% or at least 99%) identical to SEQ ID NO: 9 or a variant amino acid sequence of SEQ ID NO: 9 with 1 or 2 amino acid substitutions.

In various instances, each antibody of the antibody composition comprises: a LC variable region comprising an amino acid sequence of SEQ ID NO: 10, an amino acid sequence which is at least 90% (e.g., at least 95%, at least 96%, at least 97%, at least 98% or at least 99%) identical to SEQ ID NO: 10, or a variant amino acid sequence of SEQ ID NO: 10 with 1 to 10 (e.g., 1 to 9, 1 to 8, 1 to 7, 1 to 6, 1 to 5, 1 to 4, 1 to 3, 1 or 2) amino acid substitutions.

In exemplary aspects, each antibody of the antibody composition comprises: a HC variable region comprising an amino acid sequence of SEQ ID NO: 11, an amino acid sequence which is at least 90% (e.g., at least 95%, at least 96%, at least 97%, at least 98% or at least 99%) identical to SEQ ID NO: 11, or a variant amino acid sequence of SEQ ID NO: 11 with 1 to 10 (e.g., 1 to 9, 1 to 8, 1 to 7, 1 to 6, 1 to 5, 1 to 4, 1 to 3, 1 or 2) amino acid substitutions.

In exemplary instances, each antibody of the antibody composition comprises a light chain comprising an amino acid sequence of SEQ ID NO: 12, an amino acid sequence which is at least 90% (e.g., at least 95%, at least 96%, at least 97%, at least 98% or at least 99%) identical to SEQ ID NO: 12, or a variant amino acid sequence of SEQ ID NO: 12 with 1 to 10 (e.g., 1 to 9, 1 to 8, 1 to 7, 1 to 6, 1 to 5, 1 to 4, 1 to 3, 1 or 2) amino acid substitutions.

In various aspects, each antibody of the antibody composition comprises a heavy chain comprising an amino acid sequence of SEQ ID NO: 13, an amino acid sequence which is at least 90% (e.g., at least 95%, at least 96%, at least 97%, at least 98% or at least 99%) identical to SEQ ID NO: 13, or a variant amino acid sequence of SEQ ID NO: 13 with 1 to 10 (e.g., 1 to 9, 1 to 8, 1 to 7, 1 to 6, 1 to 5, 1 to 4, 1 to 3, 1 or 2) amino acid substitutions.

In exemplary instances, each antibody of the antibody composition comprises a light chain constant region comprising an amino acid sequence of SEQ ID NO: 14, an amino acid sequence which is at least 90% (e.g., at least 95%, at least 96%, at least 97%, at least 98% or at least 99%) identical to SEQ ID NO: 14, or a variant amino acid sequence of SEQ ID NO: 14 with 1 to 10 (e.g., 1 to 9, 1 to 8, 1 to 7, 1 to 6, 1 to 5, 1 to 4, 1 to 3, 1 or 2) amino acid substitutions.

In various aspects, each antibody of the antibody composition comprises a heavy chain constant region comprising an amino acid sequence of SEQ ID NO: 15, an amino acid sequence which is at least 90% (e.g., at least 95%, at least 96%, at least 97%, at least 98% or at least 99%) identical to SEQ ID NO: 15, or a variant amino acid sequence of SEQ ID NO: 15 with 1 to 10 (e.g., 1 to 9, 1 to 8, 1 to 7, 1 to 6, 1 to 5, 1 to 4, 1 to 3, 1 or 2) amino acid substitutions.

In exemplary aspects, the antibody composition comprises a heterogeneous mixture of different glycoforms of the antibody. In various instances, the antibody composition may be characterized in terms of its AF glycan content and/or its β-galactosylated glycan content. In various aspects, the antibody composition is described in terms of % AF glycans and/or its % β-galactosylated glycans. Optionally, the antibody composition may be characterized in terms its content of other types of glycans, e.g., high mannose glycoforms, fucosylated glycoforms, and the like.

In exemplary embodiments, the composition is combined with a pharmaceutically acceptable carrier, diluent or excipient. Accordingly, provided herein are pharmaceutical compositions comprising the recombinant glycosylated protein composition (e.g., the antibody composition or antibody binding protein composition) described herein and a pharmaceutically acceptable carrier, diluent or excipient. As used herein, the term “pharmaceutically acceptable carrier” includes any of the standard pharmaceutical carriers, such as a phosphate buffered saline solution, water, emulsions such as an oil/water or water/oil emulsion, and various types of wetting agents.

In exemplary embodiments, the antibody composition is produced by glycosylation competent cells in cell culture as described herein.

### Additional Steps

The methods disclosed herein, in various aspects, comprise additional steps. For example, in some aspects, the methods comprise one or more upstream steps or downstream steps involved in producing, purifying, and formulating a recombinant glycosylated protein, e.g., an antibody. Optionally, the downstream steps are any one of those downstream processing steps described herein or known in the art. See, e.g., Processing Steps. In exemplary embodiments, the method comprises steps for generating host cells that express a recombinant glycosylated protein (e.g., antibody). The host cells, in some aspects, are prokaryotic host cells, e.g., E. coli or Bacillus subtilis, or the host cells, in some aspects, are eukaryotic host cells, e.g., yeast cells, filamentous fungi cells, protozoa cells, insect cells, or mammalian cells (e.g., CHO cells). Such host cells are described in the art. See, e.g., Frenzel, et al., Front Immunol 4: 217 (2013) and herein under “Cells.” For example, the methods comprise, in some instances, introducing into host cells a vector comprising a nucleic acid comprising a nucleotide sequence encoding the recombinant glycosylated protein, or a polypeptide chain thereof.

In exemplary aspects, the methods comprise maintaining cells, e.g., glycosylation-competent cells in a cell culture. Accordingly, the methods may comprise carrying out any one or more steps described herein in Maintaining Cells In A Cell Culture.

In exemplary embodiments, the methods disclosed herein comprise steps for isolating and/or purifying the recombinant glycosylated protein (e.g., recombinant antibody) from the culture. In exemplary aspects, the method comprises one or more chromatography steps including, but not limited to, e.g., affinity chromatography (e.g., protein A affinity chromatography), ion exchange chromatography, and/or hydrophobic interaction chromatography. In exemplary aspects, the method comprises steps for producing crystalline biomolecules from a solution comprising the recombinant glycosylated proteins.

The methods of the disclosure, in various aspects, comprise one or more steps for preparing a composition, including, in some aspects, a pharmaceutical composition, comprising the purified recombinant glycosylated protein. Such compositions are discussed herein.

### Maintaining Cells in a Cell Culture

With regard to the methods of producing an antibody composition of the present disclosure, the antibody composition may be produced by maintaining cells in a cell culture. The cell culture may be maintained according to any set of conditions suitable for production of a recombinant glycosylated protein. For example, in some aspects, the cell culture is maintained at a particular pH, temperature, cell density, culture volume, dissolved oxygen level, pressure, osmolality, and the like. In exemplary aspects, the cell culture prior to inoculation is shaken (e.g., at 70 rpm) at 5% CO₂ under standard humidified conditions in a CO₂ incubator. In exemplary aspects, the cell culture is inoculated with a seeding density of about 10⁶ cells/mL in 1.5 L medium.

In exemplary aspects, the methods of the disclosure comprise maintaining the glycosylation-competent cells in a cell culture medium at a pH of about 6.85 to about 7.05, e.g., in various aspects, about 6.85, about 6.86, about 6.87, about 6.88, about 6.89, about 6.90, about 6.91, about 6.92, about 6.93, about 6.94, about 6.95, about 6.96, about 6.97, about 6.98, about 6.99, about 7.00, about 7.01, about 7.02, about 7.03, about 7.04, or about 7.05.

In exemplary aspects, the methods comprise maintaining the cell culture at a temperature between 30° C. and 40° C. In exemplary embodiments, the temperature is between about 32° C. to about 38° C. or between about 35° C. to about 38° C.

In exemplary aspects, the methods comprise maintaining the osmolality between about 200 mOsm/kg to about 500 mOsm/kg. In exemplary aspects, the method comprises maintaining the osmolality between about 225 mOsm/kg to about 400 mOsm/kg or about 225 mOsm/kg to about 375 mOsm/kg. In exemplary aspects, the method comprises maintaining the osmolality between about 225 mOsm/kg to about 350 mOsm/kg. In various aspects, osmolality (mOsm/kg) is maintained at about 200, 225, about 250, about 275, about 300, about 325, about 350, about 375, about 400, about 425, about 450, about 475, or about 500.

In exemplary aspects, the methods comprise maintaining dissolved the oxygen (DO) level of the cell culture at about 20% to about 60% oxygen saturation during the initial cell culture period. In exemplary instances, the method comprises maintaining DO level of the cell culture at about 30% to about 50% (e.g., about 35% to about 45%) oxygen saturation during the initial cell culture period. In exemplary instances, the method comprises maintaining DO level of the cell culture at about 20%, about 25%, about 30%, about 35%, about 40%, about 45%, about 50%, about 55%, or about 60% oxygen saturation during the initial cell culture period. In exemplary aspects, the DO level is about 35 mm Hg to about 85 mmHg or about 40 mm Hg to about 80 mmHg or about 45 mm Hg to about 75 mm Hg.

The cell culture is maintained in any one or more culture medium. In exemplary aspects, the cell culture is maintained in a medium suitable for cell growth and/or is provided with one or more feeding media according to any suitable feeding schedule. In exemplary aspects, the method comprises maintaining the cell culture in a medium comprising glucose, fucose, lactate, ammonia, glutamine, and/or glutamate. In exemplary aspects, the method comprises maintaining the cell culture in a medium comprising manganese at a concentration less than or about 1 μM during the initial cell culture period. In exemplary aspects, the method comprises maintaining the cell culture in a medium comprising about 0.25 μM to about 1 μM manganese. In exemplary aspects, the method comprises maintaining the cell culture in a medium comprising negligible amounts of manganese. In exemplary aspects, the method comprises maintaining the cell culture in a medium comprising copper at a concentration less than or about 50 ppb during the initial cell culture period. In exemplary aspects, the method comprises maintaining the cell culture in a medium comprising copper at a concentration less than or about 40 ppb during the initial cell culture period. In exemplary aspects, the method comprises maintaining the cell culture in a medium comprising copper at a concentration less than or about 30 ppb during the initial cell culture period. In exemplary aspects, the method comprises maintaining the cell culture in a medium comprising copper at a concentration less than or about 20 ppb during the initial cell culture period. In exemplary aspects, the medium comprises copper at a concentration greater than or about 5 ppb or greater than or about 10 ppb. In exemplary aspects, the cell culture medium comprises mannose. In exemplary aspects, the cell culture medium does not comprise mannose.

In exemplary embodiments, the type of cell culture is a fed-batch culture or a continuous perfusion culture. However, the methods of the disclosure are advantageously not limited to any particular type of cell culture.

The cells maintained in cell culture may be glycosylation-competent cells. In exemplary aspects, the glycosylation-competent cells are eukaryotic cells, including, but not limited to, yeast cells, filamentous fungi cells, protozoa cells, algae cells, insect cells, or mammalian cells. Such host cells are described in the art. See, e.g., Frenzel, et al., Front Immunol 4: 217 (2013). In exemplary aspects, the eukaryotic cells are mammalian cells. In exemplary aspects, the mammalian cells are non-human mammalian cells. In some aspects, the cells are Chinese Hamster Ovary (CHO) cells and derivatives thereof (e.g., CHO-K1, CHO pro-3), mouse myeloma cells (e.g., NS0, GS-NS0, Sp2/0), cells engineered to be deficient in dihydrofolatereductase (DHFR) activity (e.g., DUKX-X11, DG44), human embryonic kidney 293 (HEK293) cells or derivatives thereof (e.g., HEK293T, HEK293-EBNA), green African monkey kidney cells (e.g., COS cells, VERO cells), human cervical cancer cells (e.g., HeLa), human bone osteosarcoma epithelial cells U2-OS, adenocarcinomic human alveolar basal epithelial cells A549, human fibrosarcoma cells HT1080, mouse brain tumor cells CAD, embryonic carcinoma cells P19, mouse embryo fibroblast cells NIH 3T3, mouse fibroblast cells L929, mouse neuroblastoma cells N2a, human breast cancer cells MCF-7, retinoblastoma cells Y79, human retinoblastoma cells SO-Rb50, human liver cancer cells Hep G2, mouse B myeloma cells J558L, or baby hamster kidney (BHK) cells (Gaillet et al. 2007; Khan, Adv Pharm Bull 3(2): 257-263 (2013)).

Cells that are not glycosylation-competent can also be transformed into glycosylation-competent cells, e.g. by transfecting them with genes encoding relevant enzymes necessary for glycosylation. Exemplary enzymes include but are not limited to oligosaccharyltransferases, glycosidases, glucosidase I, glucosidease II, calnexin/calreticulin, glycosyltransferases, mannosidases, GlcNAc transferases, galactosyltransferases, and sialyltransferases.

In exemplary embodiments, the glycosylation-competent cells are not genetically modified to alter the activity of an enzyme of the de novo pathway or the salvage pathway. These two pathways of fucose metabolism are shown in FIG. 2. In exemplary embodiments, the glycosylation-competent cells are not genetically modified to alter the activity of any one or more of: a fucosyl-transferase (FUT, e.g., FUT1, FUT2, FUT3, FUT4, FUT5, FUT6, FUT7, FUT8, FUT9), a fucose kinase, a GDP-fucose pyrophosphorylase, GDP-D-mannose-4,6-dehydratase (GMD), and GDP-keto-6-deoxymannose-3,5-epimerase, 4-reductase (FX). In exemplary embodiments, the glycosylation-competent cells are not genetically modified to knock-out a gene encoding FX.

In exemplary embodiments, the glycosylation-competent cells are not genetically modified to alter the activity β(1,4)-N-acetylglucosaminyltransferase Ill (GNTIII) or GDP-6-deoxy-D-lyxo-4-hexulose reductase (RMD). In exemplary aspects, the glycosylation-competent cells are not genetically modified to overexpress GNTIII or RMD.

The following examples are given merely to illustrate embodiments of the present invention and not in any way to limit its scope.

### EXAMPLES

### Example 1

This example describes an exemplary method of determining an N-linked glycosylation profile (glycan profile) for a monoclonal antibody.

The purpose of this analytical method is to determine the N-linked glycosylation profile of an antibody in samples comprising the antibody by hydrophilic interaction liquid chromatography (HILIC) ultra high performance liquid chromatography (UHPLC) glycan map analysis. This glycan map method is a quantitative analysis of the N-linked glycan distribution of the antibody and comprises releasing and labeling N-linked glycans from reference and test samples using PNGase F and a fluorophore that can specifically derivatize free glycan, loading samples within the validated linear range onto a HILIC column, separating the labeled N-linked glycans using a gradient of decreasing organic solvent, and monitoring the elution of glycan species with a fluorescence detector.

The standard and test samples are prepared by carrying out the following: (1) dilute samples and controls with water, (2) add PNGase F and incubate the samples and controls to release N-linked glycans, (3) mix with fluorophore labeling solution using a fluorophore such as 2-aminobenzoic acid. Vortex and incubate the samples and controls, (4) centrifuge down to pellet protein and remove supernatant, and (5) dry and reconstitute labeled glycans in the injection solution.

The solutions used in this assay are a Mobile Phase A (100 mM ammonium formate, target pH 3.0) and a Mobile Phase B (acetonitrile). The equipment used to perform the method has the following capabilities:

Equipment capabilities:
HPLC system
Fluorescence detector set to appropriate
excitation/emission wavelength optimized to labeling
fluorophore
Data collection system
Temperature-controlled autosampler
Hydrophilic interaction column

The instrument settings for HPLC using a hydrophilic interaction analytical BEH Glycan 1.7 m column (2.1 mm ID×150 mm) and 2-aminobenzoic acid fluorophore labeling method are provided below:

| | | |
|---|---|---|
| Target sample load | 2 | μL |
| Column heater set point | 35° | C. |
| Auto-sampler set point | 10° | C. |

| | |
|---|---|
| Detection | Excitation 360 nm |
| | Emission 425 nm |

The mobile phase gradient example is provided below:

| Time | Flow Rate | Mobile | Mobile |
|---|---|---|---|
| (minutes) | (mL/minute) | Phase A (%) | Phase B (%) |
| 0.0 | 0.25 | 22.0 | 78.0 |
| 111.2 | 0.25 | 40.1 | 59.9 |
| 117.9 | 0.20 | 90.0 | 10.0 |
| 124.5 | 0.20 | 90.0 | 10.0 |
| 129.1 | 0.25 | 22.0 | 78.0 |
| 155 | 0.25 | 22.0 | 78.0 |

Reports of the results comprise the following format:

Report area % for β-galactosylation, high mannose and afucosylation*
% β-galactose (% β-gal) = % A2G2F + % A2G1F + %
A2G2 + % A1GG1M5 + % A2G1
% High mannose (% HM) = % M5 + % M6 + % M7
% Afucosylation (% Afuc) = % A1G0 + % A2G0 + % A2G1(a) +
% A2G1(b) + % A2G2 + % A1G1M5
*Calculation formulas depend on presence of individual high mannose and afucosylated species

An example representative glycan map chromatogram is shown in FIG. 2A (full scale view) and FIG. 2B (expanded scale view).

### Example 2

This example describes an exemplary FcγRIIa binding assay.

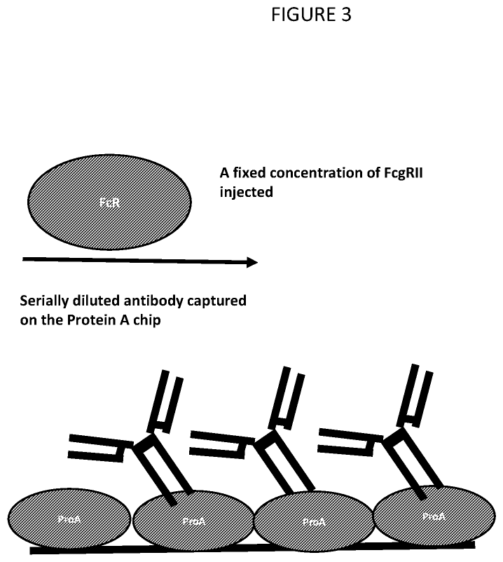

An FcγRIIa binding assay using a Biacore T200 (GE Healthcare) was developed for measuring the FcγRIIa binding activity of a sample comprising an antibody. An illustration of the assay is provided in FIG. 3. In this assay, samples comprising various concentrations of serially-diluted Antibody 1, an antibody against human complement C5 with a hybrid Fc domain of IgG2/IgG4, were used for capture on a Protein A sensor chip (Series S Sensor Chip Protein A; GE Healthcare). The binding to an isoform of FcγRIIa comprising His at amino acid position 131 (hereinafter referred to as “FcγRIIa-H”) was detected by injecting a fixed concentration of FcγRIIa-H over the surfaces comprising Protein A-captured antibody. The binding data was fitted in a linear model using a statistical software PLA 3.0 and the percent relative binding of the samples was calculated comparing to the binding levels of Antibody 1 Reference Standard (Ab1 RS).

The FcγRIIa binding assay was analyzed for method linearity, intermediate precision and accuracy. For these experiments, Ab 1 RS (49.8 mg/mL) or Ab 2 (10.1 mg/mL) were used for preparing five simulated activity samples (at 60%, 80%, 100%, 130%, and 160% levels). The sample at 100% nominal level was also used as the assay control.

The method linearity, or the ability of the method to obtain results that are directly proportional to the concentration of the analyte in the sample, was established. Five simulated binding levels (60, 80, 100, 130 and 160%) were assessed in six independent assays. A linear relationship between the expected natural log (Ln) binding levels and observed natural log binding values was demonstrated for samples with binding levels in the range of 60-160%. The values observed for slope, Y-intercept, and R² were 0.9908, 0.0473 and 0.9998 respectively.

The data generated in the linearity experiments were used to determine the intermediate precision and the accuracy of the binding assay. Intermediate precision for the method was estimated to be 1.1%, and the accuracy for the method across all binding levels was observed to be 100.5%.

The repeatability of the FcγRIIa binding assay was determined by testing four independently prepared Ab 1 RS at the 100% nominal level. Four independently prepared samples at 1× nominal concentration were tested in a total of six assays by two analysts. The overall % CV for repeatability for this assay is 1.1%.

The specificity of the FcγRIIa binding assay was also assessed as follows: Ab 1 (100 nM) was captured on Flow Cell 2 of the Protein A sensor chip, and 200 nM of FcγRIIa-H was injected over the Flow Cell 1 (without Ab 1) and the Flow Cell 2 (with Ab 1) surfaces. The sensorgrams demonstrated that FcγRIIa-H specifically binds to Ab 1 captured on the Protein A chip and only a background signal to the Protein A chip without Ab 1 was detected.

These results support that this FcγRIIa binding assay was qualified for method repeatability and linearity, precision, accuracy over the binding range of 60%-160%.

### Example 3

This example demonstrates a correlation between FcγRIIa binding and glycan content.

Antibody 1 is an antibody against human complement C5 with a hybrid Fc domain of IgG2/IgG4. Antibody 1 has the amino acid sequence of eculizumab, an antibody approved in the U.S. and Europe for the treatment of Paroxysmal Noctumal Hemoglobinuria (PIN) and atypical Hemolytic Uremic Syndrome (aHUS). The glycan profile for different samples comprising Antibody 1 were determined following the procedure described in Example 1. These samples were also characterized in terms of their FcγRIIa binding activity by carrying out the assay described in Example 2.

Table 1 lists the measured amounts of high mannose (HM) glycans, β-galactosylated glycans, and afucosylated glycans as well as the measured FcγRIIa binding activity (expressed as % relative binding).

TABLE 1

| Sample | | β- | | Measured | Predicted |
|---|---|---|---|---|---|
| ID | | galacto- | Afuco- | FcyRlla | FcyRlla |
| No. | HM | sylated | sylated | binding | binding |
| 1 | 4.2 | 19.7 | 1.9 | 96.0 | 94 |
| 2 | 5.5 | 13.5 | 2.4 | 87.0 | 88 |
| 3 | 5.8 | 13.5 | 2.7 | 86.0 | 86 |
| 5 | 5.5 | 34.1 | 1.1 | 105.0 | 106 |
| 6 | 3.9 | 19.5 | 1.0 | 100.0 | 100 |
| 7 | 5.5 | 23.8 | 1.1 | 100.0 | 101 |
| 8 | 5.6 | 34.1 | 1.1 | 106.0 | 106 |
| 9 | 3.9 | 19.5 | 1.0 | 100.0 | 100 |
| 10 | 5.6 | 34.1 | 1.0 | 106.0 | 106 |
| 11 | 4.1 | 18.8 | 1.8 | 95.0 | 94 |
| 12 | 4.7 | 16.5 | 2.0 | 91.0 | 92 |

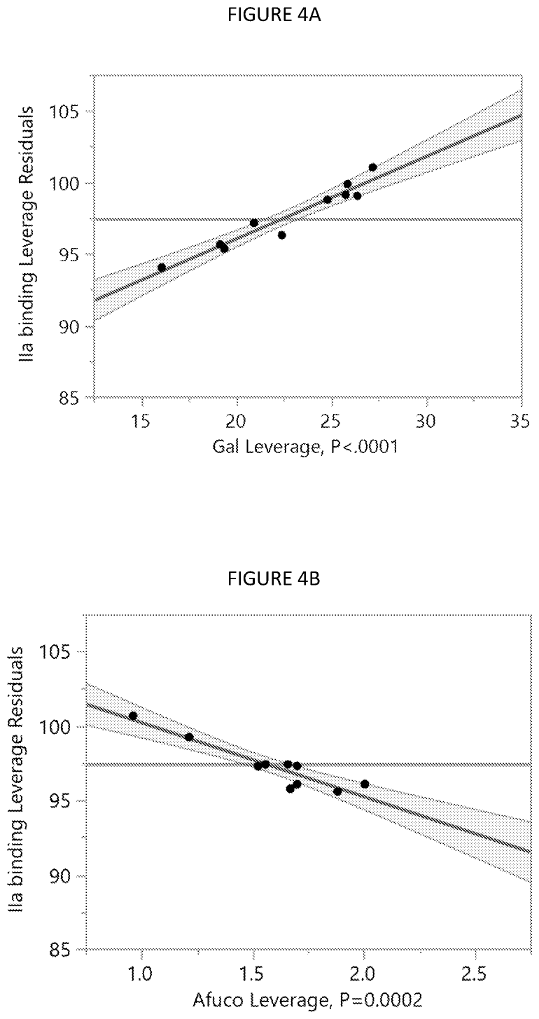

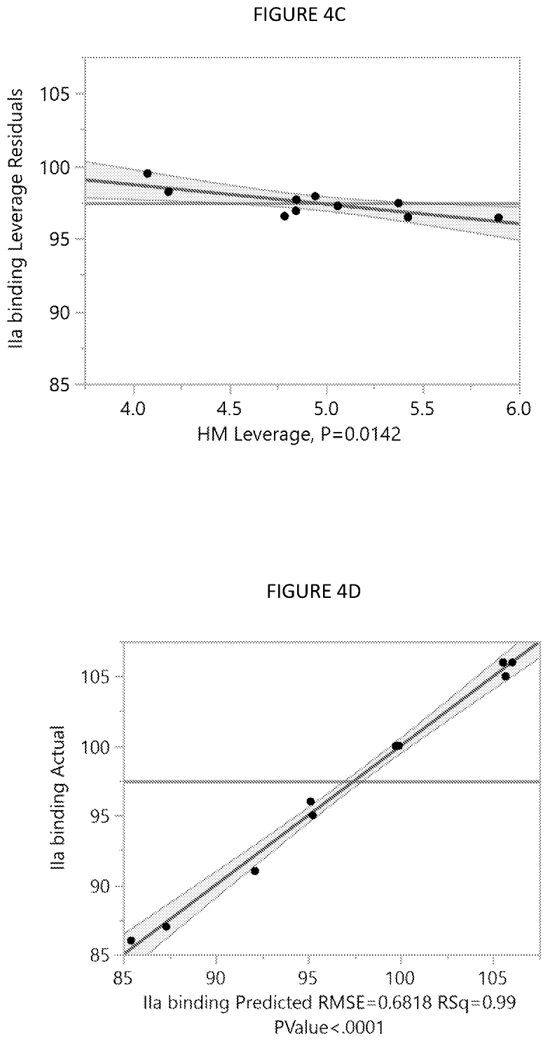

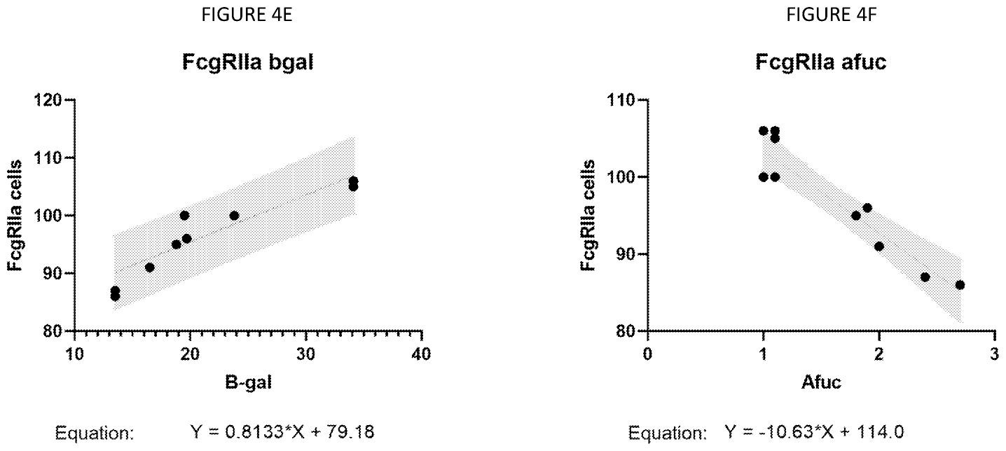

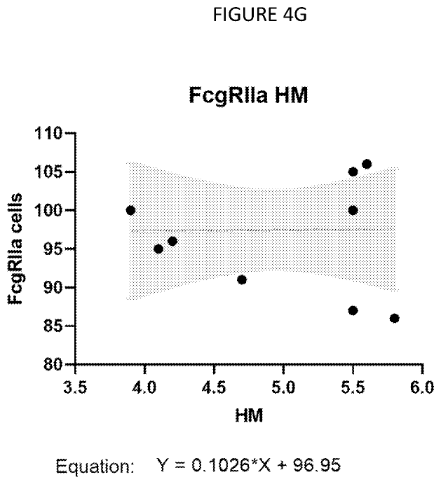

The data for measured FcγRIIa binding, HM content. β-galactosylated content and afucosylated glycan content were analyzed using the JMP suite of computer programs for statistical analysis (SAS Institute, Cary, NC). The results are shown in FIGS. 4A to 4C, wherein FIG. 4A is an FcγRIIa binding leverage plot for β-galactosylated glycans, FIG. 4B is a leverage plot for afucosylated glycans, and FIG. 4C is a leverage plot for HM glycans. The best fit line of each graph is shown as a dark red line. As shown in these figures, each of the β-galactosylated content, afucosylated glycan content and the high mannose content associated with FcγRIIa binding and each association was statistically significant (p<0.0001 for β-galactosylated glycans; p=0.0002 for afucosylated glycans; and p=0.0142 for high mannose). The relationship between % FcγRIIa binding and β-galactosylated content, afucosylated glycan content, and high mannose content for Antibody 1 may be described by Equation 1:

$$[ Equation 1 ]$$

$$Predicted \% FcyRIIa binding = 98.877 + ( 0.576 * \% β - Galactosylated Glycans ) + ( 4.978 * \% Afucosylated Glycans + ( - 1.343 \% High Mannose Glycans )$$

Plugging the measured values for % β-galactosylated glycans, % afucosylated glycans, and high mannose glycans into Equation 1, a predicted % FcγRIIa binding value was calculated for each sample and provided in Table 1. The actual % FcγRIIa binding (as measured in the FcγRIIa binding assay) was plotted against the predicted % FcγRIIa binding (as calculated by Equation 1) and the plot is provided as FIG. 4D. FIG. 4D also provides statistical parameters, including Root Mean Square Error (RMSE), r² (RSq) and p-value. These results suggest that Equation 1 predicted the actual (measured) FcγRIIa binding with accuracy and underlines the statistically significant direct correlation between β-galactosylated glycans, afucosylated glycans, high mannose glycans and FcγRIIa binding (p<0.0001). Higher levels of β-galactosylated glycans and lower levels of afucosylated glycans and high mannose glycans result in higher FcγRIIa binding activity. The leverage of β-galactosylated glycans and the leverage of afucosylated glycans were highly similar (p<0.0001 and p<0.0002, respectively).

The data for measured FcγRIIa binding, β-galactosylated content, afucosylated glycan content and HM content were also analyzed using the GraphPad Prism software for statistical analysis (GraphPad, San Diego, CA). The results are shown in FIGS. 4E-4G and in each figure, the equation of the regression line (shown as the dashed line) and the 95% confidence interval (shown by the light blue area) are shown. As shown in these figures, most data points fell within the 95% confidence interval for FIGS. 4E and 4F. As shown in FIG. 4G, the confidence interval is much broader than the those of FIGS. 4E and 4F. The range of slopes and y-intercepts for the equations of FIGS. 4E and 4F are provided below in Table 2.

TABLE 2

| FIG. | Equation* | Range of slopes | Range of y-intercepts |
|---|---|---|---|
| 4E | Y = 0.8133X + 79.18 | 0.5352 to 1.091 | 72.58 to 85.78 |
| 4F | Y = (−10.53)X + 114 | −13.73 to −7.540 | 108.8 to 119.1 |

*Y = predicted % FcyRlla binding. X = % glycan indicated in the figure. Each equation is in the form of y = mx + b, wherein m is slope and b is y-intercept.

Based on the dataset used in this study, FcγRIIa binding of an antibody composition may be predicted by measuring the β-galactosylated content, afucosylated glycan content and/or HM content. The data support a strong impact of the β-galactosylated content and afucosylated glycan content on FcγRIIa binding. Accordingly, the FcγRIIa binding of an antibody composition may be predicted by measuring just the β-galactosylated content and afucosylated glycan content. The data of FIGS. 4E-4F support that FcγRIIa binding may be predicted reasonably well by measuring just the β-galactosylated content of the afucosylated glycan content. Data points within the 95% confidence intervals would reasonably predict FcγRIIa binding of an antibody composition.

### Example 4

This example describes an exemplary FcγRIIb binding assay.

An FcγRIIb binding assay using a Biacore T200 (GE Healthcare) was developed for measuring the FcγRIIb binding activity of a sample comprising an antibody. In this assay, samples comprising various concentrations of serially-diluted Antibody 1 were used for capture on a Protein A sensor chip (Series S Sensor Chip Protein A; GE Healthcare). The binding to an human FcγRIIb-GST-H6 recombinant protein (hereinafter referred to as “FcγRIIB-GST-H6”) was detected by injecting a fixed concentration of FcγRIIb-GST-H6 over the surfaces comprising Protein A-captured antibody. The binding data was fitted in a linear model using a statistical software PLA 3.0 and the percent relative binding of the samples was calculated comparing to the binding levels of Test Antibody 1 Reference Standard (Ab1 RS).

### Example 5

This example demonstrates a correlation between FcγRIIb binding and glycan content.

The glycan profile for different samples comprising Antibody 1 were determined following the procedure described in Example 1. These samples were also characterized in terms of their FcγRIIb binding activity by carrying out the assay described in Example 4.

Table 3 lists the measured amounts of high mannose (HM) glycans, β-galactosylated glycans, and afucosylated glycans as well as the measured FcγRIIb binding activity (expressed as % relative binding).

TABLE 3

| Sample | | β- | | Measured | Predicted |
|---|---|---|---|---|---|
| ID | | galacto- | Afuco- | FcyRlla | FcyRlla |
| No. | HM | sylated | sylated | binding | binding |
| 1 | 4.2 | 19.7 | 1.9 | 99.0 | 97 |
| 2 | 5.5 | 13.5 | 2.4 | 91.0 | 92 |
| 3 | 5.8 | 13.5 | 2.7 | 89.0 | 90 |
| 4 | 5.5 | 34.1 | 1.1 | 108.0 | 106 |
| 5 | 3.9 | 19.5 | 1.0 | 105.0 | 103 |
| 6 | 5.5 | 23.8 | 1.1 | 103.0 | 103 |
| 7 | 5.6 | 34.1 | 1.1 | 105.0 | 107 |
| 8 | 3.9 | 19.5 | 1.0 | 100.0 | 103 |
| 9 | 5.6 | 34.1 | 1.0 | 105.0 | 107 |
| 10 | 4.1 | 18.8 | 1.8 | 100.0 | 97 |
| 11 | 4.7 | 16.5 | 2.0 | 94.0 | 95 |

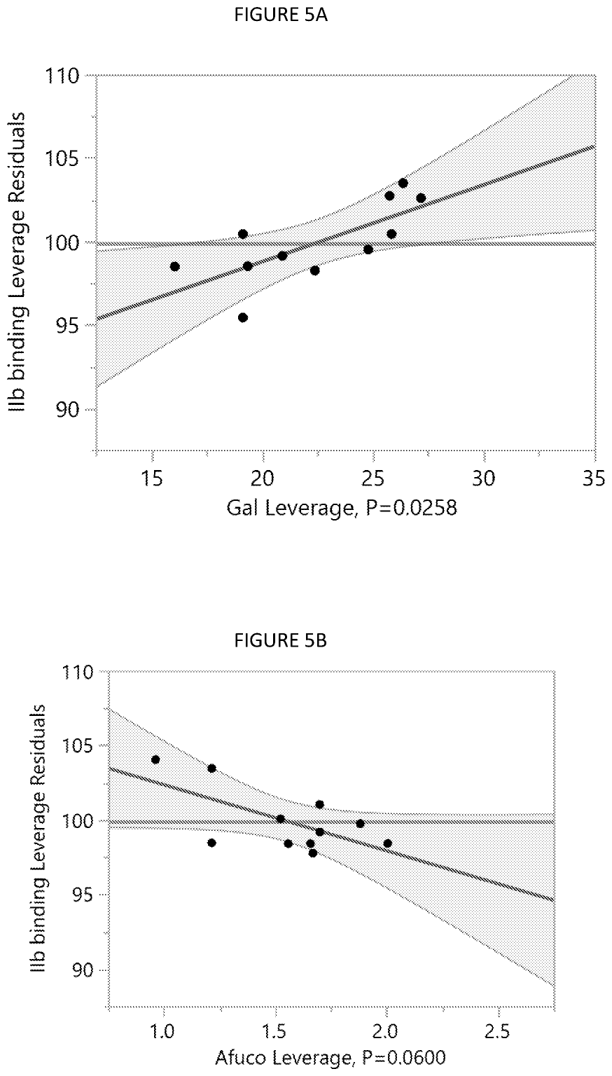

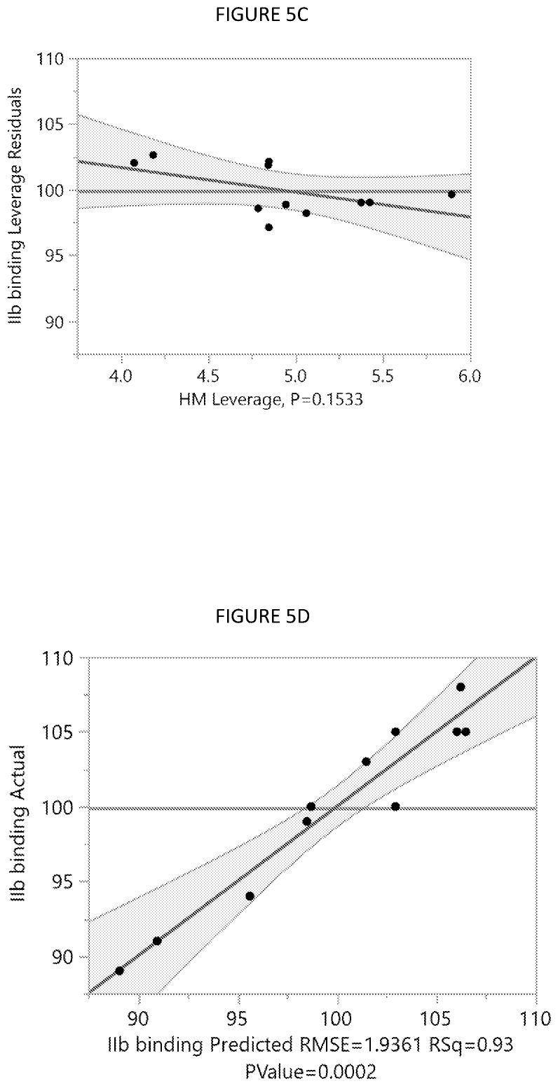

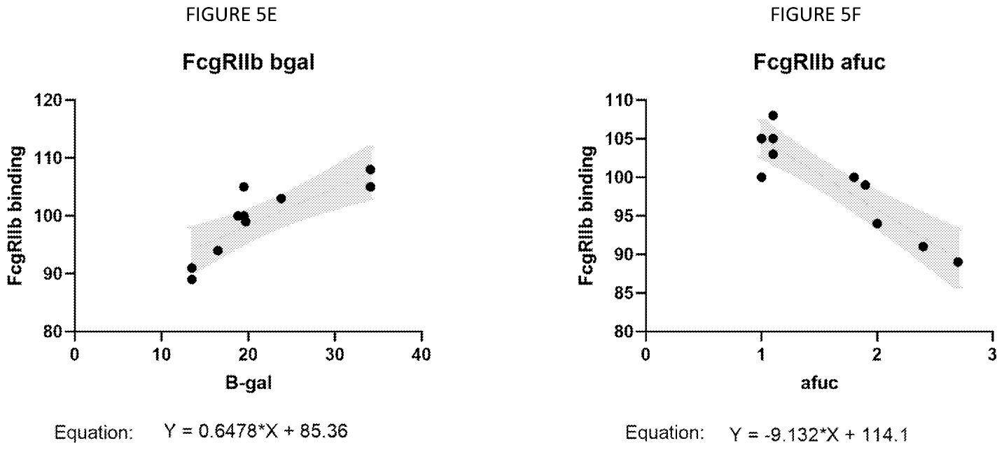

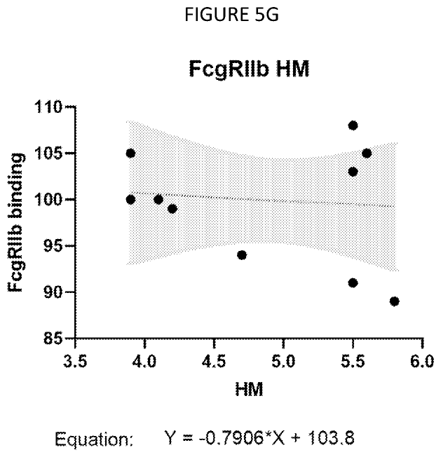

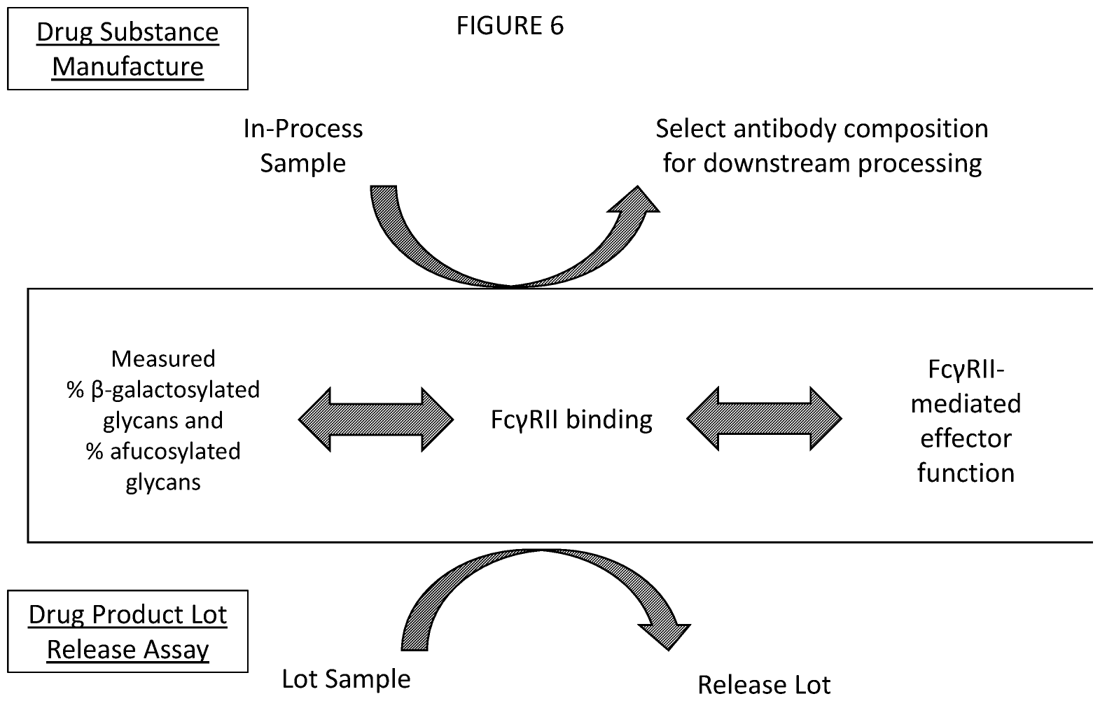

The data for measured FcγRIIb binding, high mannose content, β-galactosylated content and afucosylated glycan content were analyzed using the JMP suite of computer programs for statistical analysis (SAS Institute, Cary, NC). The results are shown in FIGS. 5A to 5C, wherein FIG. 5A is an FcγRIIb binding leverage plot for β-galactosylated glycans, FIG. 5B is a leverage plot for afucosylated glycans, and FIG. 5C is a leverage plot for HM glycans. The best fit line of each graph is shown as a dark red line. As shown in these figures, β-galactosylated content and afucosylated glycan content associated with FcγRIIb binding and each association was statistically significant (p=0.0258 for β-galactosylated glycans; p=0.0600 for afucosylated glycans). As shown in FIG. 5C, the association between HM content and FcγRIIb binding was not statistically significant (p=0.1533). The relationship between % FcγRIIb binding and β-galactosylated content and afucosylated glycan content for Antibody 1 may be described by Equation 2:

$$[ Equation 2 ]$$

$$Predicted \% FcyRIIb binding = 105.731 + ( 0.461 * \% β - Galactosylated Glycans ) + ( - 4.429 * \% Afucosylated Glycans + ( - 1.883 ) \% HM Glycans )$$

Plugging the measured values for % β-galactosylated glycans and % afucosylated glycans into Equation 2, a predicted % FcγRIIb binding value was calculated for each sample and provided in Table 3. The actual % FcγRIIb binding (as measured in the FcγRIIb binding assay) was plotted against the predicted % FcγRIIb binding (as calculated by Equation 2) and the plot is provided as FIG. 5D. FIG. 5D also provides statistical parameters, including Root Mean Square Error (RMSE), r², and p-value. These results suggest that Equation 2 predicted the actual (measured) FcγRIIb binding with accuracy and underlines the clear correlation between β-galactosylated glycans, afucosylated glycans, and FcγRIIb binding. Higher levels of β-galactosylated glycans and lower levels of afucosylated glycans result in higher FcγRIIb binding activity. The leverage of β-galactosylated glycans and the leverage of afucosylated glycans were similar (p=0.0258 and p=0.0600, respectively).

The data for measured FcγRIIb binding, HM content, β-galactosylated content and afucosylated glycan content were also analyzed using the GraphPad Prism software for statistical analysis (GraphPad, San Diego, CA). The results are shown in FIGS. 5E-5G and in each figure, the equation of the regression line (shown as the dashed line) and the 95% confidence interval (shown by the light blue area) are shown. As shown in these figures, most data points fell within the 95% confidence interval for FIGS. 5E and 5F. The range of slopes and y-intercepts for the equations of FIGS. 5E and 5F are provided below in Table 4.

TABLE 4

| FIG. | Equation* | Range of slopes | Range of y-intercepts |
|---|---|---|---|
| 5E | Y = 0.6478X + 85.36 | 0.3260 to 0.9697 | 77.72 to 92.99 |
| 5F | Y = (−9.132)X + 114 | −12.02 to −6.247 | 109.3 to 118.9 |

*Y = predicted % FcyRlla binding. X = % glycan indicated in the figure. Each equation is in the form of y = mx + b, wherein m is slope and b is y-intercept.

Based on the dataset used in this study, FcγRIIb binding of an antibody composition may be predicted by measuring the β-galactosylated content and afucosylated glycan content. The data of FIGS. 5E-5F support that FcγRIIb binding may be predicted with reasonable confidence by measuring just the β-galactosylated content of the afucosylated glycan content. Data points within the 95% confidence intervals would reasonably predict FcγRIIb binding of an antibody composition.

### Example 6

This example demonstrates a correlation between FcγRII binding and glycan content.

The experiments and analyses of Examples 1-5 were carried out with additional samples comprising Antibody 1. Briefly, glycan profiles of the samples comprising Antibody 1 were determined following the procedures described in Example 1. These samples were also characterized in terms of their FcγRIIa binding activity and FcγRIIb binding activity by carrying out the assays described in Examples 2 and 4, respectively.

Table 5 lists the measured amounts of high mannose (HM) glycans, β-galactosylated (β-gal) glycans, and afucosylated (afuco) glycans as well as the measured FcγRIIa binding activity and FcγRIIb binding activity (each expressed as % relative binding) for previously analyzed samples (Sample ID Nos: 1-11) and additional samples (Sample ID Nos. 12-19).

TABLE 5

| | | | | Measured | Predicted | Measured | Predicted |
|---|---|---|---|---|---|---|---|
| Sample | | | | FcγRIIa | FcγRIIa | FcγRIIb | FcγRIIb |
| ID No. | HM | β-gal | Afuco | binding | binding | binding | binding |
| 1 | 4.2 | 19.7 | 1.9 | 96 | 96 | 99 | 99 |
| 2 | 5.5 | 13.5 | 2.4 | 87 | 88 | 91 | 92 |
| 3 | 5.8 | 13.5 | 2.7 | 86 | 86 | 89 | 91 |
| 4 | 3.6 | 15.9 | 2.2 | 98 | 94 | 99 | 97 |
| 5 | 3 | 31.5 | 1.4 | 104 | 108 | 110 | 109 |
| 6 | 4.2 | 17.9 | 2 | 96 | 95 | 99 | 98 |
| 7 | 5.5 | 34.1 | 1.1 | 105 | 105 | 108 | 107 |
| 8 | 3.9 | 19.5 | 1 | 100 | 101 | 105 | 101 |
| 9 | 5.5 | 23.8 | 1.1 | 100 | 100 | 103 | 101 |
| 10 | 5.6 | 34.1 | 1.1 | 106 | 105 | 105 | 106 |
| 11 | 3.8 | 17.3 | 0.9 | 101 | 100 | 99 | 100 |
| 12 | 3.5 | 21.5 | 1 | 104 | 103 | 101 | 103 |
| 13 | 3.8 | 24.2 | 1 | 103 | 104 | 101 | 104 |
| 14 | 3.9 | 17.1 | 1.9 | 93 | 96 | 98 | 98 |
| 15 | 4.1 | 18.8 | 1.8 | 95 | 97 | 100 | 99 |
| 16 | 4.7 | 16.5 | 2 | 91 | 93 | 94 | 96 |
| 17 | 4.2 | 20.7 | 2 | 100 | 97 | 100 | 99 |
| 18 | 3 | 31.8 | 1.3 | 112 | 108 | 107 | 110 |
| 19 | 2.6 | 33 | 1.4 | 107 | 109 | 112 | 111 |

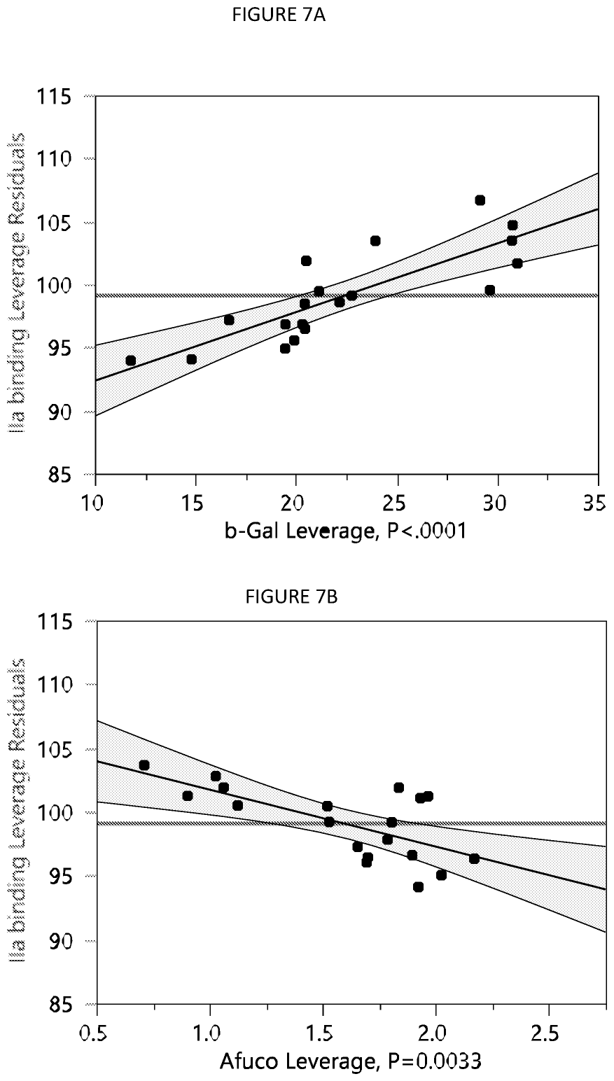

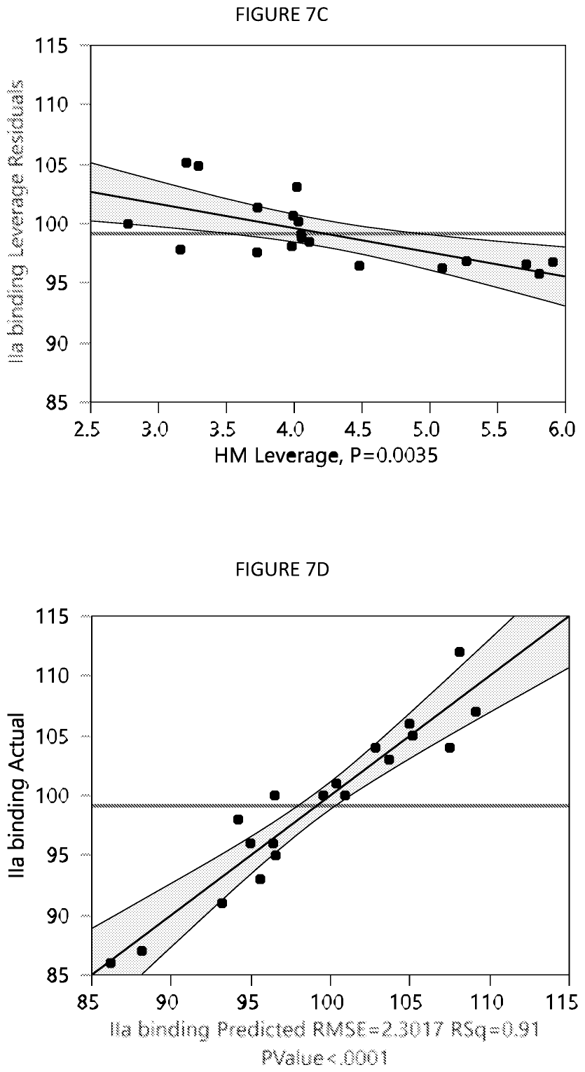

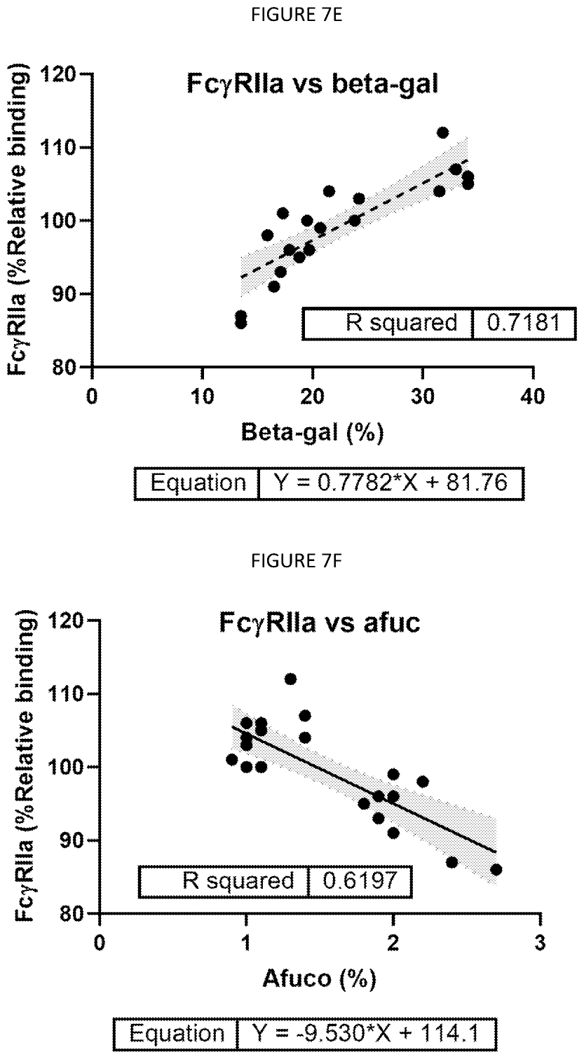

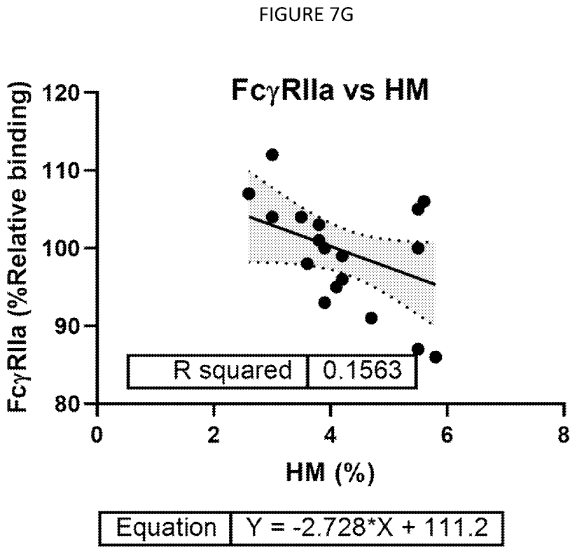

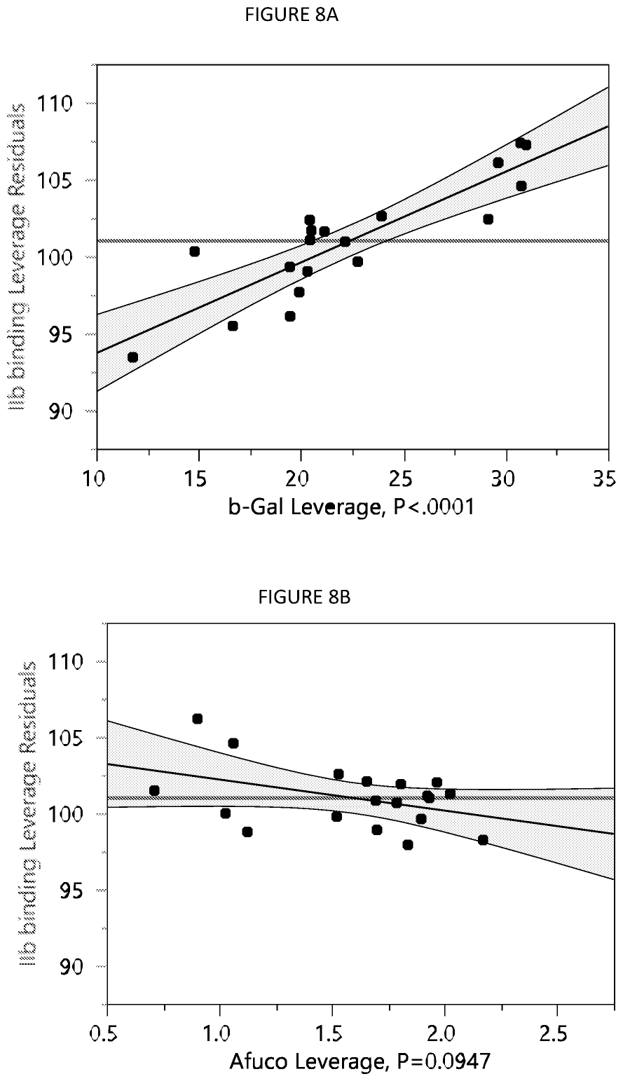

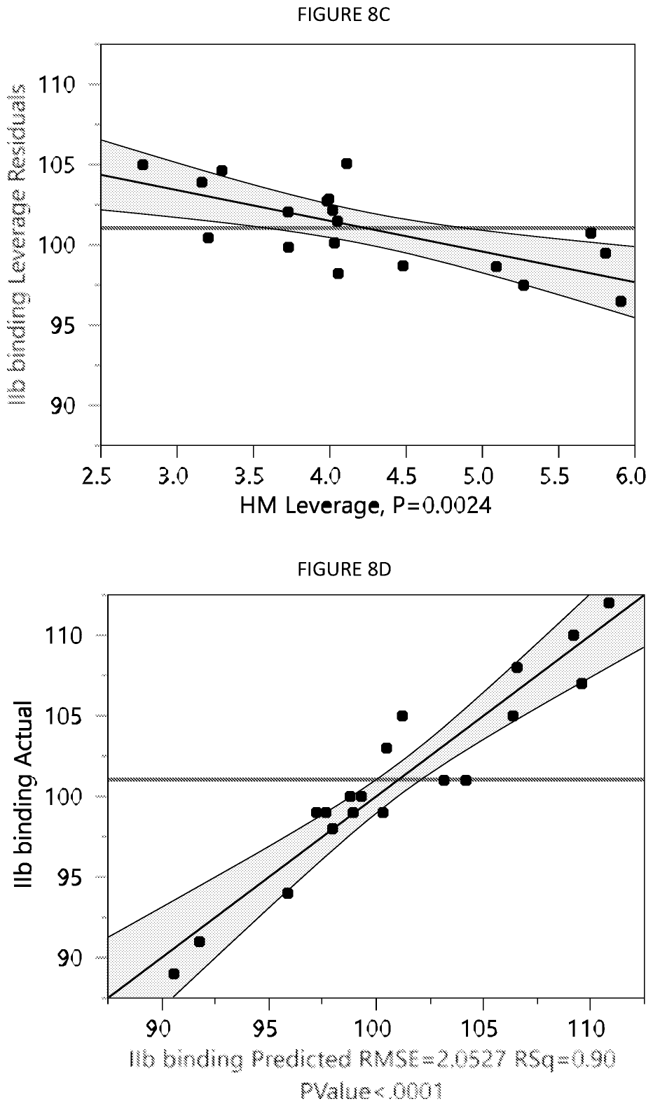

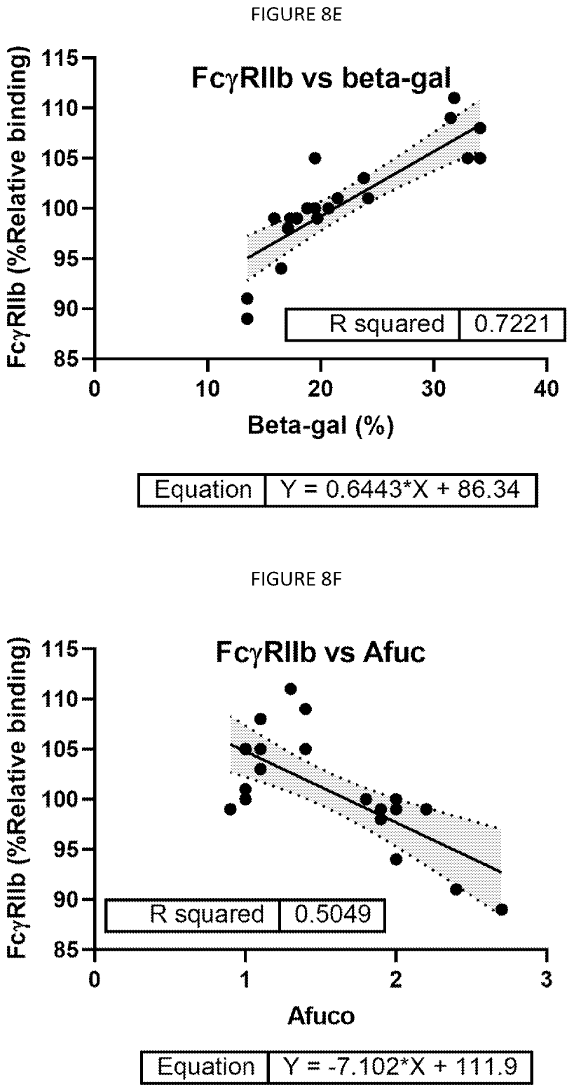

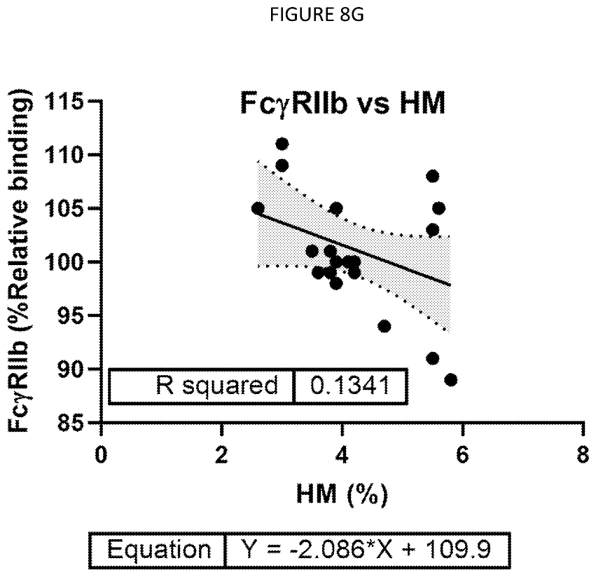

The data for measured FcγRIIa binding, measured FcγRIIb binding, high mannose content, β-galactosylated content and afucosylated glycan content of Table 5 were analyzed using the JMP suite of computer programs for statistical analysis (SAS Institute, Cary, NC). The results are shown in FIGS. 7A to 7C and FIGS. 8A to 8C, wherein FIG. 7A is an FcγRIIa binding leverage plot for β-galactosylated glycans, FIG. 7B is FcγRIIa binding leverage plot for afucosylated glycans, FIG. 7C is an FcγRIIa binding leverage plot for HM glycans, FIG. 8A is an FcγRIIb binding leverage plot for R-galactosylated glycans, FIG. 8B is an FcγRIIb binding leverage plot for afucosylated glycans, and FIG. 8C is an FcγRIIb binding leverage plot for HM glycans. The best fit line of each graph is shown as a dark red line.

As shown in FIGS. 7A-7B, β-galactosylated content and afucosylated glycan content associated with FcγRIIa binding and each association was statistically significant (p<0.0001 for β-galactosylated glycans; p=0.0033 for afucosylated glycans). As shown in FIG. 7C, the association between HM content and FcγRIIa binding was also statistically significant (p=0.0035). The relationship between % FcγRIIa binding and β-galactosylated content, afucosylated glycan content, and HM content for Antibody 1 may be described by Equation 7:

$$[ Equation 7 ]$$

$$Predicted \% FcyRIIa binding = 102.704 + ( 0.545 * \% β - Galactosylated Glycans ) + ( - 4.466 * \% Afucosylated Glycans + ( - 2.0356 ) \% HM Glycans )$$

Plugging the measured values for % β-galactosylated glycans, % afucosylated glycans, and HM glycans of Table 5 into Equation 7, a predicted % FcγRIIa binding value was calculated for each sample. The predicted % FcγRIIa binding value is also presented in Table 5. The actual % FcγRIIa binding (as measured in the FcγRIIa binding assay) was plotted against the predicted % FcγRIIa binding (as calculated by Equation 7) and the plot is provided as FIG. 7D. FIG. 7D also provides statistical parameters, including Root Mean Square Error (RMSE), r², and p-value. These results support that Equation 7 predicted the actual (measured) FcγRIIa binding with accuracy and underlines the clear correlation between β-galactosylated glycans, afucosylated glycans, HM glycans and FcγRIIa binding. Higher levels of β-galactosylated glycans and lower levels of afucosylated glycans and HM glycans result in higher FcγRIIa binding activity. The leverage of each glycan group was similar to one another.

As shown in FIGS. 8A and 8C, β-galactosylated content and HM glycan content associated with FcγRIIb binding and each association was statistically significant (p<0.0001 for β-galactosylated glycans; p=0.0024 for HM glycans). As shown in FIG. 8B, the afucosylated glycan content trended with FcγRIIb binding (p=0.0947). The relationship between % FcγRIIb binding and β-galactosylated content, afucosylated glycan content, and HM content for Antibody 1 may be described by Equation 8:

$$[ Equation 8 ]$$

$$Predicted \% FcyRIIb binding = 99.211 + ( 0.59 * \% β - Galactosylated Glycans ) + ( - 2.04 * \% Afucosylated Glycans + ( - 1.911 ) \% HM Glycans )$$

Plugging the measured values for % β-galactosylated glycans, % afucosylated glycans, and HM glycans of Table 5 into Equation 8, a predicted % FcγRIIb binding value was calculated for each sample. The predicted % FcγRIIb binding value is also presented in Table 5. The actual % FcγRIIb binding (as measured in the FcγRIIb binding assay) was plotted against the predicted % FcγRIIb binding (as calculated by Equation 8) and the plot is provided as FIG. 8D. FIG. 8D also provides statistical parameters, including Root Mean Square Error (RMSE), r², and p-value. These results support that Equation 8 predicted the actual (measured) FcγRIIb binding with accuracy and underlines the clear correlation between β-galactosylated glycans, afucosylated glycans, HM glycans and FcγRIIb binding. Higher levels of β-galactosylated glycans and lower levels of afucosylated glycans and HM glycans result in higher FcγRIIb binding activity. The leverage of each glycan group was similar to one another.

The data for measured FcγRIIa binding and measured FcγRIIb binding, measured HM content, β-galactosylated content and afucosylated glycan content of Table 5 were additionally analyzed using the GraphPad Prism software for statistical analysis (GraphPad, San Diego, CA). The results are shown in FIGS. 7E-7G and 8E-8G, wherein FIG. 7E is a graph plotting FcγRIIa binding as a function of β-galactosylated content, FIG. 7F is a graph plotting FcγRIIa binding as a function of afucosylated content, FIG. 7G is a graph plotting FcγRIIa binding as a function of HM content, FIG. 8E is a graph plotting FcγRIIb binding as a function of β-galactosylated content, FIG. 8F is a graph plotting FcγRIIb binding as a function of afucosylated content, and FIG. 8G is a graph plotting FcγRIIb binding as a function of HM content. In each figure, the equation of the regression line (shown as the dashed line) and the 95% confidence interval (shown by the light blue area) are shown.

Based on the dataset used in this study, FcγRIIa binding of an antibody composition may be predicted by measuring the β-galactosylated content, afucosylated glycan content, and HM content. The data of FIGS. 7E-7G support that FcγRIIa binding may be predicted with reasonable confidence by measuring these glycans. Data points within the 95% confidence intervals would reasonably predict FcγRIIa binding of an antibody composition. Similar observations were made for measured FcγRIIb binding and the glycan content. Based on the dataset used in this study, FcγRIIb binding of an antibody composition may be predicted by measuring the β-galactosylated content, afucosylated glycan content, and HM content. The data of FIGS. 8E-8G support that FcγRIIb binding may be predicted with reasonable confidence by measuring these glycans. Data points within the 95% confidence intervals would reasonably predict FcγRIIb binding of an antibody composition.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. A method of determining product quality of an antibody composition, wherein the product quality is based on the Fcγ receptor II (FcγRII) binding level of the antibody composition, said method comprising
a. determining the afucosylated glycan content and/or β-galactosylated glycan content of a sample of the antibody composition;
b. optionally, calculating a FcγRII binding level based on the afucosylated glycan content and/or β-galactosylated glycan content as determined in (a); and
c. determining the product quality of the antibody composition as acceptable when (i) the afucosylated glycan content and/or β-galactosylated glycan content is within a target range and/or (ii) the FcγRII binding level is within a target range.

2. The method of claim 1, wherein the target range of FcγRII binding levels, the target range of the afucosylated glycan content and/or the target range of the β-galactosylated glycan content is based on the FcγRII binding levels, the afucosylated glycan content, and/or the β-galactosylated glycan content of a reference antibody.

3. The method of claim 2, wherein the reference antibody comprises a chimeric constant region.

4. The method of claim 2 or 3, wherein the reference antibody comprises a portion of an IgG2 constant region and a portion of an IgG4 constant region, optionally, wherein the reference antibody is eculizumab.

5. The method of any one of the preceding claims, wherein the FcγRII binding level is a level of FcγRIIa binding.

6. The method of any one of the preceding claims, comprising calculating a FcγRIIa binding level based on the β-galactosylated glycan content determined in (a).

7. The method of claim 6, wherein the FcγRII binding level is calculated according to Equation A:
$$FcyRII binding level = m * \% BG + y , \qquad [ Equation A ]$$
wherein m is about 0.535 to about 1.091, y is about 72.58 to about 85.78, and % BG is the % β-galactosylated glycan content determined in (a).

8. The method of claim 7, wherein m is 0.813 or 0.778.

9. The method of claim 7 or 8, wherein y is 79.18 or 81.76.

10. The method of any one of the preceding claims, comprising calculating a FcγRII binding level based on the afucosylated glycan content determined in (a).

11. The method of claim 8, wherein the FcγRII binding level is calculated according to Equation B:
$$FcyRII binding level = m * \% AF + y , \qquad [ Equation B ]$$
wherein m is about −13.73 to about −7.54, y is about 108.8 to about 119.1, and % AF is the % afucosylated glycan content determined in (a).

12. The method of claim 11, wherein m is −10.63 or −9.53.

13. The method of claim 11 or 12, wherein y is 114.

14. The method of any one of the preceding claims, comprising calculating a FcγRII binding level based on the afucosylated glycan content and the β-galactosylated glycan content determined in (a).

15. The method of claim 14, wherein the FcγRII binding level is within the 95% confidence interval of a line of Equation 3:
$$FcyRII binding = 0.576 * \% BG + ( - 4.978 ) * \% AF + 98.877 , \qquad [ Equation 3 ]$$
wherein % BG is the % β-galactosylated glycan content and % AF is the % afucosylated glycan content.

16. The method of any one of the preceding claims, wherein the FcγRII binding level is a level of FcγRIIb binding.

17. The method of any one of the preceding claims, comprising calculating a FcγRIIb binding level based on the β-galactosylated glycan content determined in (a).

18. The method of claim 17, wherein the FcγRII binding level is calculated according to Equation C:
$$FcyRII binding level = m * \% BG + y , \qquad [ Equation C ]$$
wherein m is about 0.3260 to about 0.9697, y is about 77.72 to about 92.99, and % BG is the % β-galactosylated glycan content determined in (a).

19. The method of claim 18, wherein m is 0.648 or 0.644.

20. The method of claim 18 or 19, wherein y is 85.36 or 86.34.

21. The method of any one of the preceding claims, comprising calculating a FcγRIIb binding level based on the afucosylated glycan content determined in (a).

22. The method of claim 21, wherein the FcγRII binding level is calculated according to Equation D:
$$FcyRII binding level = m * \% AF + y , \qquad [ Equation D ]$$
wherein m is about −12.02 to about −6.247, y is about 109.3 to about 118.9, and % AF is the % afucosylated glycan content determined in (a).

23. The method of claim 22, wherein m is −9.132 or −7.102.

24. The method of claim 22 or 23, wherein y is 114 or 111.9.

25. The method of any one of the preceding claims, comprising calculating a FcγRIIb binding level based on the afucosylated glycan content and the β-galactosylated glycan content determined in (a).

26. The method of claim 25, wherein the FcγRII binding level is within the 95% confidence interval of a line of Equation 4:
$$FcyRII binding = 0.461 * \% BG + ( - 4.429 ) * \% AF + 105.731 . \qquad [ Equation 4 ]$$

27. The method of any one of the preceding claims, further comprising determining the high mannose (HM) glycan content.

28. The method of claim 27, wherein the FcγRII binding level is within the 95% confidence interval of a line of Equation 5 or Equation 6 or Equation 9 or Equation 10:
$$[ Equation 5 ]$$
$$FcyRIIa binding = 0.576 * \% BG + ( - 4.978 ) * \% AF + 98.877 + ( - 1.343 ) * \% HM$$
$$[ Equation 6 ]$$
$$FcyRIIa binding = 0.461 * \% BG + ( - 4.429 ) * \% AF + 105.731 + ( - 1.883 ) * \% HM$$
$$[ Equation 9 ]$$
$$FcyRII binding = 0.545 * \% BG + ( - 4.466 ) * \% AF + 102.7 + ( - 2.036 ) * \% HM ,$$
$$[ Equation 10 ]$$
$$FcyRII binding = 0.59 * \% BG + ( - 2.04 ) * \% AF + 99.2 + ( - 1.91 ) * \% HM ,$$
wherein % BG is the % β-galactosylated glycan content, % AF is the % afucosylated glycan content, and % HM is the % high mannose glycan content.

29. The method of any one of the preceding claims, wherein the method is a quality control (QC) assay.

30. The method of any one of the preceding claims, wherein the method is an in-process QC assay.

31. The method of any one of the preceding claims, wherein the sample is a sample of in-process material.

32. The method of any one of the preceding claims, wherein the afucosylated glycan content and/or β-galactosylated glycan content is determined pre-harvest or post-harvest.

33. The method of any one of the preceding claims, wherein the afucosylated glycan content and/or β-galactosylated glycan content is determined after a chromatography step.

34. The method of claim 33, wherein the chromatography step comprises a capture chromatography, intermediate chromatography, and/or polish chromatography.

35. The method of claim 33 or 34, wherein the afucosylated glycan content and/or β-galactosylated glycan content is determined after a virus inactivation and neutralization, virus filtration, or a buffer exchange.

36. The method of any one of the preceding claims, wherein the method is a lot release assay.

37. The method of any one of the preceding claims, wherein the sample is obtained from a manufacturing lot.

38. The method of any one of the preceding claims, further comprising selecting the antibody composition for downstream processing, when the afucosylated glycan content and/or β-galactosylated glycan content is within a target range.

39. The method of any one of the preceding claims, wherein, when the afucosylated glycan content and/or β-galactosylated glycan content determined in (a) is not within the target range, one or more conditions of the cell culture are modified to obtain a modified cell culture.

40. The method of claim 39, further comprising determining the afucosylated glycan content and/or β-galactosylated glycan content of a sample of the antibody composition obtained after one or more conditions of the cell culture are modified.

41. The method of any one of the preceding claims, wherein, when the afucosylated glycan content and/or β-galactosylated glycan content determined in (a) is not within the target range, the method further comprises (d) modifying one or more conditions of the cell culture to obtain a modified cell culture and (e) determining the afucosylated glycan content and/or β-galactosylated glycan content of a sample of the antibody composition obtained from the modified cell culture.

42. The method of claim 41, wherein, when the afucosylated glycan content and/or β-galactosylated glycan content determined in (a) is not within the target range, the method further comprises (d) and (e) until the afucosylated glycan content and/or β-galactosylated glycan content determined in (e) is within the target range.

43. The method of any one of the preceding claims, wherein an assay which directly measures FcγRII binding level of the antibody composition is carried out on the antibody composition only when the the afucosylated glycan content and/or β-galactosylated glycan content is outside the target range.

44. The method of any one of the preceding claims wherein an assay which directly measures FcγRII binding of the antibody composition is not carried out on the antibody composition when the afucosylated glycan content and/or β-galactosylated glycan content is within the target range.

45. A method of monitoring product quality of an antibody composition, comprising determining product quality of an antibody composition in accordance with a method of any one of the preceding claims, with a first sample obtained at a first timepoint and with a second sample taken at a second timepoint which is different from the first timepoint.

46. The method of claim 45, wherein each of the first sample and second sample is a sample of in-process material.

47. The method of claim 46, wherein the first sample is a sample of in-process material and the second sample is a sample of a manufacturing lot.

48. The method of claim 46, wherein the first sample is a sample obtained before one or more conditions of the cell culture are modified and the second sample is a sample obtained after the one or more conditions of the cell culture are modified.

49. A method of producing an antibody composition, comprising determining the product quality of the antibody composition, wherein product quality of the antibody composition is determined in accordance with a method of any one of claims 1-44, wherein the sample is a sample of in-process material, wherein, when the afucosylated glycan content and/or β-galactosylated glycan content determined in (a) is not within the target range, the method further comprises (d) modifying one or more conditions of the cell culture to obtain a modified cell culture and (e) determining the afucosylated glycan content and/or β-galactosylated glycan content of a sample of the antibody composition obtained from the modified cell culture, optionally, repeating steps (d) and (e) until the afucosylated glycan content and/or β-galactosylated glycan content is within the target range.

50. The method of claim 49, wherein one or more conditions of the cell culture are modified to primarily change the HM glycan content to achieve the target range of FcγRII binding.

51. The method of claim 50, wherein one or more conditions of the cell culture are modified to primarily change the β-galactosylated glycan content to achieve the target range of FcγRII binding.

52. A method of producing an antibody composition, comprising
a. determining the afucosylated glycan content and/or β-galactosylated glycan content of a sample of the antibody composition;
b. determining the FcγRII binding level of the antibody composition based on afucosylated glycan content and/or β-galactosylated glycan content determined in (a); and
c. selecting the antibody composition for downstream processing based on the level of FcγRII binding determined in (b).

53. The method of claim 52, wherein the antibody composition comprises a chimeric constant region.

54. The method of claim 53, wherein the chimeric constant region comprises a portion of an IgG2 constant region and a portion of an IgG4 constant region.

55. The method of any one of claims 3-54, wherein the chimeric constant region comprises CH1 of an IgG2 and/or CH2-CH3 of an IgG4.

56. The method of claim 55, wherein the antibody composition comprises a chimeric constant region comprising an amino acid sequence of SEQ ID NO: 15.

57. The method of any one of the preceding claims, wherein the antibody composition comprises an anti-C5 antibody comprising the heavy chain and light chain of eculizumab.

58. The method of any one of the preceding claims, wherein the sample is of a cell culture comprising glycosylation-competent cells expressing an antibody of the antibody composition.

59. The method of claim 58, further comprising modifying one or more conditions of the cell culture to modify the afucosylated glycan content and/or the β-galactosylated glycan content of the antibody composition and determining the afucosylated glycan content and/or the β-galactosylated glycan content of a sample of the antibody composition taken from the modified cell culture.

60. The method of claim 59, further comprising modifying one or more conditions of the cell culture to increase the level of afucosylated glycans of the antibody composition to decrease the level of FcγRII binding of the antibody composition.

61. The method of claim 59 or 60, further comprising modifying one or more conditions of the cell culture to decrease the level of β-galactosylated glycans of the antibody composition to decrease the level of FcγRII binding of the antibody composition.

62. The method of claim 59, further comprising modifying one or more conditions of the cell culture to decrease the level of afucosylated glycans of the antibody composition to increase the level of FcγRII binding of the antibody composition.

63. The method of claim 59 or 62, further comprising modifying one or more conditions of the cell culture to increase the level of β-galactosylated glycans of the antibody composition to increase the level of FcγRII binding of the antibody composition.

64. The method of any one of claims 59 to 63, further comprising repeating said modifying until the afucosylated glycan content and/or the β-galactosylated glycan content is within a target range.

65. The method of any one of the preceding claims, wherein the afucosylated glycan content and/or the β-galactosylated glycan content is/are determined in real time with respect to production of the antibody composition.

66. The method of any one of the preceding claims, comprising selecting the antibody composition for downstream processing when the afucosylated glycan content and/or the β-galactosylated glycan content is/are in a target range.

67. The method of any one of the preceding claims, comprising selecting the antibody composition for downstream processing when the FcγRII binding level is in a target range.

68. The method of any one of the preceding claims, wherein determining the level of FcγRII binding comprises determining a level of ADCC, ADCP, and/or CDC.

69. The method of any one of the preceding claims, further comprising specifying a level of ADCC, ADCP, and/or CDCC of the antibody composition, wherein the selected antibody composition comprises the specified level of ADCC, ADCP, and/or CDC

70. A method of modifying the level of FcγRII binding of an antibody composition, comprising
a) specifying a level of FcγRII; and
b) modifying the level of afucosylated glycans and/or β-galactosylated glycans of the antibody composition to achieve the specified level of FcγRII.

71. The method of claim 70, comprising increasing the level of afucosylated glycans of the antibody composition to decrease the level of FcγRII binding of the antibody composition.

72. The method of claim 70 or 71, comprising decreasing the level of β-galactosylated glycans of the antibody composition to decrease the level of FcγRII binding of the antibody composition.

73. The method of claim 70, comprising decreasing the level of afucosylated glycans of the antibody composition to increase the level of FcγRII binding of the antibody composition.

74. The method of claim 70 or 73, comprising increasing the level of β-galactosylated glycans of the antibody composition to increase the level of FcγRII binding of the antibody composition.

75. A method of determining the level of Fcγ receptor II (FcγRII) binding of an antibody composition, comprising determining the level of afucosylated glycans and/or β-galactosylated glycans of the antibody composition.

76. A method of predicting in vivo efficacy and/or adverse effects of an antibody composition, comprising
a. determining the afucosylated glycan content and/or β-galactosylated glycan content of a sample of the antibody composition;
b. predicting the antibody composition as causative of in vivo adverse effects based on the afucosylated glycan content and/or β-galactosylated glycan content determined in (a).

77. The method of any one of the preceding claims, wherein the FcγRII is FcγRIIa.

78. The method of any one of the preceding claims, wherein the FcγRII is FcγRIIb.

79. The method of any one of the preceding claims, wherein the antibody composition comprises a chimeric constant region.

80. The method of claim 79, wherein the chimeric constant region comprises a portion of an IgG2 constant region and a portion of an IgG4 constant region.

81. The method of claim 80, wherein the chimeric constant region comprises CH1 of an IgG2 and/or CH2-CH3 of an IgG4.

82. The method of claim 81, wherein the antibody composition comprises a chimeric constant region comprising an amino acid sequence of SEQ ID NO: 15.

83. The method of any one of the preceding claims, wherein the antibody comprises eculizumab.

84. The method of any one of the preceding claims, wherein the downstream processing comprises at least one of dilution, concentration, filling, filtration, formulation, chromatography, viral filtration, and/or viral inactivation.

85. The method of any one of the preceding claims, wherein the downstream processing comprises chromatography such as capture chromatography, intermediate chromatography, and/or polish chromatography.

86. The method of claim 85, wherein the chromatography comprises one or more of affinity chromatography, ion exchange chromatography, or hydrophobic interaction chromatography.
